(19) United States
(12) Patent Application Publication
(10) Pub. No.: US 2025/0090786 A1
(43) Pub. Date: Mar. 20, 2025

(54) SYSTEMS AND METHODS FOR GENERATING CONCENTRATED OXYGEN

(71) Applicant: VENTEC LIFE SYSTEMS, INC., Bothell, WA (US)

(72) Inventors: Laurent BROUQUEYRE, Marietta, GA (US); Andrew R. CHAPMAN, Bothell, WA (US); Shan E. GAW, Seattle, WA (US); Adam SMITH, Palm Desert, CA (US)

(21) Appl. No.: 18/726,713

(22) PCT Filed: Jan. 3, 2023
(86) PCT No.: PCT/US2023/060051

§ 371 Date: Jul. 3, 2024

Related U.S. Application Data
(60) Provisional application No. 63/266,403, filed on Jan. 4, 2022.

(51) Int. Cl. A61M 16/10; B01D 53/04; B01D 53/047

(52) U.S. Cl. CPC A61M 16/101; B01D 53/0415; B01D 53/047; B01D 2256/12; B01D 2257/102; B01D 2257/80; B01D 2259/4533

(57) ABSTRACT

The present technology is directed to systems and methods for generating concentrated oxygen for therapeutic purposes. For example, in some embodiments the systems described herein include an oxygen assembly that can provide pulses of oxygen and/or a continuous flow of oxygen to a patient. The oxygen assembly can include one or more media or adsorption beds configured to generate concentrated oxygen from ambient air, such as by removing nitrogen from ambient air flowing through the media bed. The one or more media beds can be removed from the system to facilitate replacement of the media bed by a user. The oxygen assemblies and media beds described herein can also include various additional features that are expected to improve the oxygen generation process and/or the operation of the oxygen generating systems.

### CROSS-REFERENCE TO RELATED APPLICATION(S)

The present application claims priority to U.S. Provisional Patent Application No. 63/266,403, filed Jan. 4, 2022, and titled “SYSTEMS AND METHODS FOR GENERATING CONCENTRATED OXYGEN,” the disclosure of which is incorporated by reference herein in its entirety.

### TECHNICAL FIELD

The present technology is generally directed to systems and methods for generating concentrated oxygen for therapeutic use.

### BACKGROUND

Concentrated oxygen is used to treat a wide variety of medical conditions in a wide variety of patients. For example, some patients may require oxygen therapy while at home, some patients may require oxygen therapy while ambulatory, etc. These and other patients often use a portable oxygen concentrator. Such oxygen concentrators are convenient because they can generate concentrated oxygen from ambient air and therefore do not need to be connected to an external (e.g., high pressure) oxygen supply. However, conventional oxygen concentrators may demonstrate a limited lifespan, high power requirements, and other inefficiencies. Accordingly, a need exists for improved systems for generating concentrated oxygen.

### BRIEF DESCRIPTION OF THE DRAWINGS

Many aspects of the present technology can be better understood with reference to the following drawings. The components in the drawings are not necessarily to scale. Instead, emphasis is placed on clearly illustrating the principles of the present technology.

FIG. 1 is a schematic diagram of a ventilator and a patient circuit, each of which is configured in accordance with embodiments of the present technology.

FIGS. 2A-2D are a series of perspective views illustrating another ventilator configured in accordance with embodiments of the present technology.

FIGS. 3A and 3B are perspective views of another ventilator configured in accordance with embodiments of the present technology.

FIG. 3C is a front perspective view of an adsorption bed assembly removed from the ventilator of the ventilator of FIGS. 3A and 3B.

FIG. 3D is another front perspective view of the adsorption bed assembly of FIG. 3C with select aspects of the adsorption bed assembly omitted to better show additional features of the adsorption bed assembly.

FIG. 3E is a rear perspective view of the adsorption bed assembly of FIG. 3C.

FIG. 3F is yet another front perspective view of the adsorption bed assembly of FIG. 3C with select aspects of the adsorption bed assembly omitted to better show additional features of the adsorption bed assembly.

FIGS. 3G and 3H are perspective views of another adsorption bed assembly configured in accordance with embodiments of the present technology.

FIGS. 3I and 3J are perspective views of another adsorption bed assembly configured in accordance with embodiments of the present technology.

FIGS. 4A and 4B are schematic illustrations of oxygen assemblies including adsorption beds and configured in accordance with embodiments of the present technology.

FIGS. 5A-5D are a series of partially schematic views illustrating adsorption beds including helical inserts and configured in accordance with embodiments of the present technology.

FIGS. 6A-6C are a series of schematic views illustrating operation of an adsorption bed during an oxygen generation process and configured in accordance with embodiments of the present technology.

FIGS. 7A and 7B are schematic illustrations of an oxygen assembly including an adsorption bed having variable flow paths and configured in accordance with embodiments of the present technology.

FIGS. 8A-8D illustrate adsorption beds having a tapered flow path and configured in accordance with embodiments of the present technology.

FIG. 9 is a graph illustrating volume and pressure characteristics for the tapered adsorption beds of FIGS. 8A-8D.

### DETAILED DESCRIPTION

The present technology is directed to systems and methods for generating concentrated oxygen for therapeutic purposes. For example, in some embodiments the systems described herein include an oxygen assembly that can provide pulses of oxygen and/or a continuous flow of oxygen to the patient. The oxygen assembly can include one or more media or adsorption beds configured to generate concentrated oxygen from ambient air, such as by removing nitrogen from ambient air flowing through the media bed. As described in greater detail herein, in some embodiments the one or more media beds can be removed from the system to facilitate replacement of the media bed by a user. The oxygen assemblies and media beds described herein can also include various additional features that are expected to improve the oxygen generation process and/or the operation of the oxygen generating systems. For example, the systems described herein may demonstrate one or more of increased efficiency, increased oxygen output, more consistent oxygen output, increased lifespan, decreased power requirements, or other advantages. Accordingly, further aspects and advantages of the devices, methods, and uses will become apparent from the ensuing description that is given by way of example only.

As one skilled in the art will appreciate from the following description, the oxygen generating systems described herein include both standalone oxygen concentrators and ventilator systems with integrated oxygen production. Accordingly, in some embodiments the various features described below can be implemented to improve the performance of conventional oxygen concentrators. In other embodiments, the various features described herein can be integrated into a ventilator system that is configured to provide both ventilation therapy and oxygen therapy. For example, the systems described herein may include a ventilation assembly in addition to the oxygen assembly. The ventilation assembly can provide inspiratory gas to support the patient's breathing, in addition to or in lieu of the oxygen therapy. Accordingly, the present technology includes both standalone oxygen concentrators and ventilator systems that include an integrated oxygen concentrator.

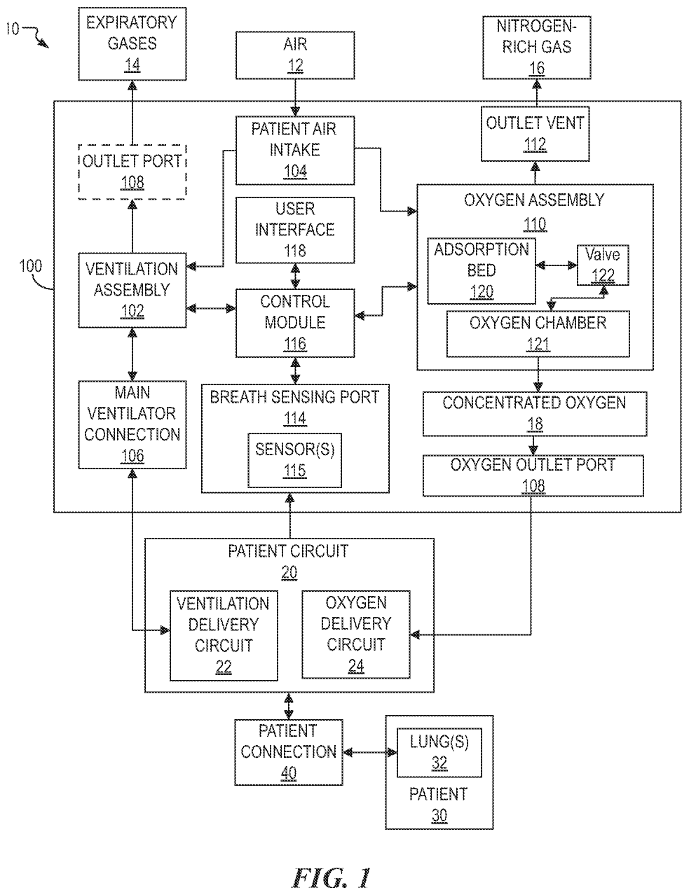

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to FIGS. 1-9.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%. The term “substantially” or grammatical variations thereof refers to at least about 50%, for example, 75%, 85%, 95%, or 98%.

The headings provided herein are for convenience only and do not interpret the scope or meaning of the claimed present technology.

### I. Select Embodiments of Ventilator Systems with Integrated Oxygen Production

FIG. 1 is block diagram of a system 10 including a ventilator 100 and a patient circuit 20 configured in accordance with embodiments of the present technology and for delivering ventilation therapy and/or oxygen therapy to a patient 30. The ventilator 100 may be configured to be portable and powered by an internal battery (not shown) and/or an external power source (not shown) such as a conventional wall outlet. The ventilator 100 can include a ventilation assembly 102 for providing ventilation gases (e.g., “air 12”) to the patient 30. The air 12 is received by the ventilator 100 via a patient air intake 104, which is coupled to the ventilation assembly 102. While identified as being “air,” those of ordinary skill in the art appreciate that the air 12 may include ambient air or pressurized air obtained from any source external to the ventilator 100. The ventilation assembly 102 can provide the air 12 to a main ventilator connection 106 during an inspiratory phase and/or an expiratory phase of a breath. In some embodiments, the ventilator 100 may receive expiratory gases during an expiratory phase of a breath. The ventilator 100 may therefore have an outlet port 107 coupled to the ventilation assembly 102 for venting patient expiratory or exhalation gases 14 to an environment external to the ventilator 100. In some embodiments, some or all of the patient expiratory or exhalation gases 14 may be vented at another suitable location of the system 10 rather than being routed back to the ventilator 100, such as via an exhaust valve (not shown) positioned at a patient connection 40 or along the patient circuit 20, both of which are described in detail below.

The ventilator 100 can also include an oxygen assembly 110 (which may also be referred to herein as an “oxygen generation assembly” or “oxygen generation module”) for providing concentrated oxygen 18 to the patient 30. The oxygen assembly 110 can include an adsorption bed 120 (which can also be referred to as an “adsorber,” “media bed,” “sieve bed,” or the like) that contains a nitrogen-adsorbent material (e.g., zeolite). The adsorption bed 120 can therefore preferentially adsorb nitrogen from the air 12 received by the oxygen assembly 110 via the patient air intake 104 (although shown as receiving air via the same patient air intake 104 as the ventilation assembly 102, in other embodiment the oxygen assembly 110 can be coupled to a separate air intake). The nitrogen-adsorbent material can be regenerated during a desorption phase in which the oxygen 18 or another gas flows through the adsorption bed 120 and purges the adsorbed nitrogen from the nitrogen-adsorbent material in the form of a nitrogen-rich gas 16, described below. As also described in greater detail below, the adsorption bed 120 can be releasably received by the ventilator 100 (e.g., the oxygen assembly 110 of the ventilator 100), such that the adsorption bed 120 can be removed and replaced (e.g., by the patient 30 and/or another user). Moreover, although described as a single adsorption bed 120, the oxygen assembly 110 may include one or more adsorption beds 120, such as two, three, four, etc.

The oxygen assembly 110 can therefore generate concentrated oxygen 18 within the ventilator 100 by flowing air 12 through the adsorption bed 120. As used herein, the term “concentrated oxygen” refers to a gas that contains between 80% and 96% pure oxygen (O2). The oxygen assembly 110 can operate using a pressure swing adsorption (PSA) process, a vacuum pressure swing adsorption (VPSA) process, or via another suitable technique. The oxygen assembly 110 can optionally include an oxygen chamber 121 for at least temporarily holding concentrated oxygen 18 generated by the adsorption bed 120. In some embodiments, the oxygen assembly 110 can further include a valve 122 (e.g., a regulator) positioned between the adsorption bed 120 and the oxygen chamber 121 for controlling the flow of oxygen 18 therebetween. For example, during an adsorption phase in which concentrated oxygen 18 is produced by flowing air 12 through the adsorption bed 120, the valve 122 can permit the oxygen 18 to flow out of the adsorption bed 120 and into the oxygen chamber 121 only after a sufficient pressure within the adsorption bed 120 has been reached, thereby ensuring the oxygen 18 has reached a sufficient purity. Likewise, during a desorption phase in which nitrogen is desorbed and purged from the adsorption bed 120 by flowing concentrated oxygen 18 or another gas through the adsorption bed 120, the valve 122 can permit oxygen 18 to flow from the oxygen chamber 121 back into and through the adsorption bed 120 to purge nitrogen from the adsorption bed 120. In some embodiments, the oxygen assembly 110 can also include certain additional features for further controlling operation of the adsorption bed 120, such as additional valving, flow conduits or the like, including those described in U.S. Pat. No. 10,046,134, the disclosure of which is incorporated by reference herein in its entirety and for all purposes. The ventilator 100 may output exhaust gases (e.g., nitrogen-rich gas 16 produced during the desorption phase by flowing oxygen 18 through the adsorption bed 120) from the oxygen assembly 110 and to an environment external to the ventilator 100 via an outlet vent 112 coupled to the oxygen assembly 110.

The ventilator 100 can be coupled to the patient 30 via the patient circuit 20 and the patient connection 40. The patient circuit 20 can include a ventilation gas delivery circuit 22 and an oxygen delivery circuit 24. The ventilation gas delivery circuit 22 is configured to be fluidly coupled to the main ventilator connection 106, and can include a conduit or lumen (e.g., tubing) for transporting gases (e.g., the air 12 during the inspiratory phase and patient exhalation gases 14 during an expiratory phase) to and/or from the patient 30. The oxygen delivery circuit 24 is configured to be fluidly coupled to an oxygen outlet port 108 and can also include a conduit or lumen (e.g., tubing) for transporting concentrated oxygen 18 to and/or from the patient 30. In some embodiments, the ventilation gas delivery circuit 22 and the oxygen delivery circuit 24 are fluidly isolated along an entirety or substantial portion of their respective lengths, such that concentrated oxygen 18 from the oxygen assembly 110 and the air 12 from the ventilation assembly 102 do not mix until at or proximate the patient connection 40. Accordingly, the ventilation gas delivery circuit 22 and the oxygen delivery circuit 24 can be formed by a multi-lumen tube having a co-axial arrangement, two or more separate tubes adjoined together, and/or two distinct circuits not coupled together. In some embodiments, the patient circuit 20 can be a passive patient circuit or an active patient circuit, such as those described in U.S. Pat. Nos. 10,518,059 and 10,105,509, the disclosures of which are incorporated by reference herein in their entireties and for all purposes. The patient circuit 20 can also be connected to a breath sensing port 114, described below. The patient connection 40 can be any suitable interface coupled to the patient circuit 20 for delivering the air 12 and concentrated oxygen 18 to the patient 30, such as a full rebreather mask, a partial rebreather mask, a nasal mask, a mouthpiece, a nose piece (e.g., a cannula), a tracheal tube, or the like. In some embodiments, the system 10 can optionally include one or more bacteria filters positioned in-line between the ventilator 100 and the patient 30, such as within the patient circuit 20 or at another suitable location.

The breath sensing port 114 can include one or more transducers or sensors 115 for measuring one or more parameters of a patient's breath and/or within the system 10 (e.g., flow, pressure, volume, etc.). For example, the breath sensing port 114 can be coupled to a portion of the patient circuit 20 configured to transmit patient signals (e.g., pressure signals) to the one or more sensors 115. The one or more sensors 115 can measure the transmitted patient signal, which can be used to trigger delivery of the breathing gases through the main ventilator connection 106 and/or pulses of the concentrated oxygen 18 through the oxygen outlet port 108 (e.g., to synchronize delivery of breathing gases and/or oxygen to patient inspiratory efforts). In some embodiments, the breath sensing port 114 can be a multi-lumen tube connection, such as the multi-lumen tube connection described in U.S. Pat. Nos. 10,245,406 and 10,315,002, the disclosures of which are incorporated by reference herein in their entireties and for all purposes.

In some embodiments, the ventilator 100 can deliver oxygen to the patient 30 independent of the ventilation therapy and/or in combination with the ventilation therapy, as described in U.S. Patent Application Publication No. US 2022/0193354, the disclosure of which is incorporated by reference herein in its entirety. For example, the system 10 can operate in an oxygen mode in which it provides pulses of supplemental concentrated oxygen 18 to the patient 30, a ventilation mode in which it provides inspiratory gases (e.g., air 12) to the patient 30, and/or a combination mode in which it provides both inspiratory gases (e.g., air 12) and pulses of concentrated oxygen 18 to the patient 30. Accordingly, the ventilator 100 can provide supplemental oxygen to the patient 30, can provide mechanical ventilation to the patient 30, and/or can provide mechanical ventilation in combination with concentrated oxygen 18 to the patient 30.

The ventilator 100 may include a control module 116 for controlling operation of the ventilator 100. For example, the control module 116 can generate one or more signals for controlling operation of the ventilation assembly 102 and/or the oxygen assembly 110. For example, the control module 116 can transition the ventilator 100 between the ventilation mode, the oxygen mode, and/or the combination mode. This may be done automatically or in response to a user input. The control module 116 can also synchronize operation of the ventilator 100 with the patient's breath. For example, in some embodiments, the control module 116 receives one or more measured parameters from the sensor(s) 115 at the breath sensing port 114. The ventilator 100 may therefore be configured to provide volume-controlled ventilation, pressure-controlled ventilation, and/or flow-controlled ventilation. For example, the control module 116 can analyze the measured parameter(s) received from the breath sensing port 114 and, based on the analysis, trigger delivery of a breath via the patient circuit 20 and/or trigger delivery of a pulse of concentrated oxygen 18 via the oxygen delivery circuit 24. The control module 116 can also be configured to control operation of the ventilator 100 without requiring a triggering signal (e.g., for patients that do not demonstrate detectable spontaneous breathing). In such embodiments, the control module 116 can operate the ventilator 100 according to preset timing or other suitable techniques. The control module 116 may also receive feedback signals from the ventilation assembly 102 and/or the oxygen assembly 110 to monitor and/or control the various aspects of the ventilator 100.

The ventilator 100 can further include a user interface 118. The user interface 118 is configured to receive input from a user (e.g., the patient 30 or a caregiver, a clinician, or the like associated with the patient 30) and provide that input to the control module 116. The input received via the user interface 118 can include ventilator settings, parameters of operation, modes of operation, and the like. In a particular example, a user may select between the ventilation mode, the oxygen mode, and/or the combination mode using the user interface 118. The user interface 118 can further be configured to display information to the user and/or patient, including ventilator settings, parameters of operation, modes of operation, status of the adsorption bed 120, estimated remaining life of the adsorption bed 120, an alert to replace the adsorption bed 120, and the like. The user interface 118 can be any suitable user interface known in the art, such as a touch-screen having a digital display.

The ventilator 100 can optionally include additional functions beyond the ventilation and oxygen delivery described herein. For example, the ventilator 100 can optionally include a nebulizer connection for coupling to a nebulizer assembly, a suction connection for coupling to a suction assembly, and/or a cough-assist module for providing cough assistance to the patient, as described in U.S. Pat. No. 9,956,371, the disclosure of which is incorporated by reference herein in its entirety.

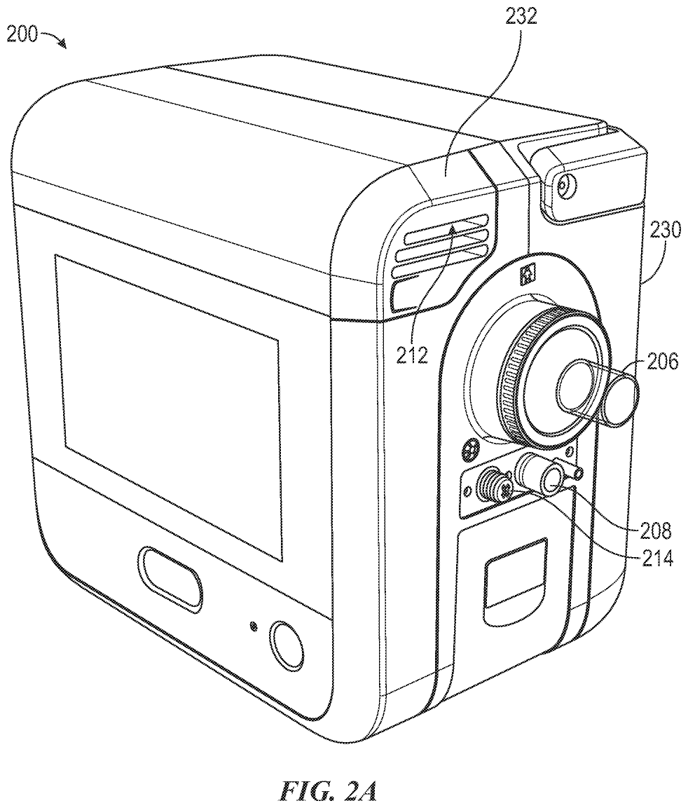

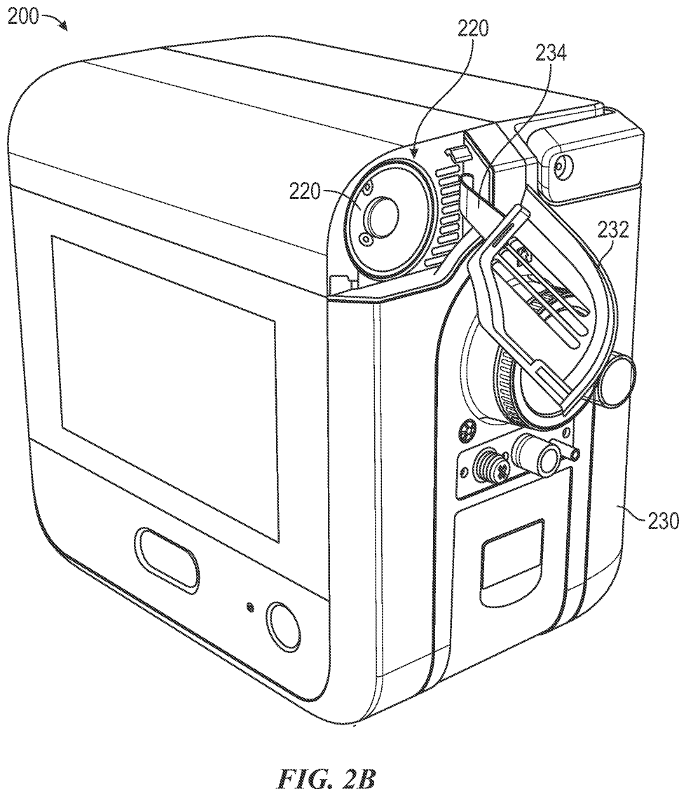

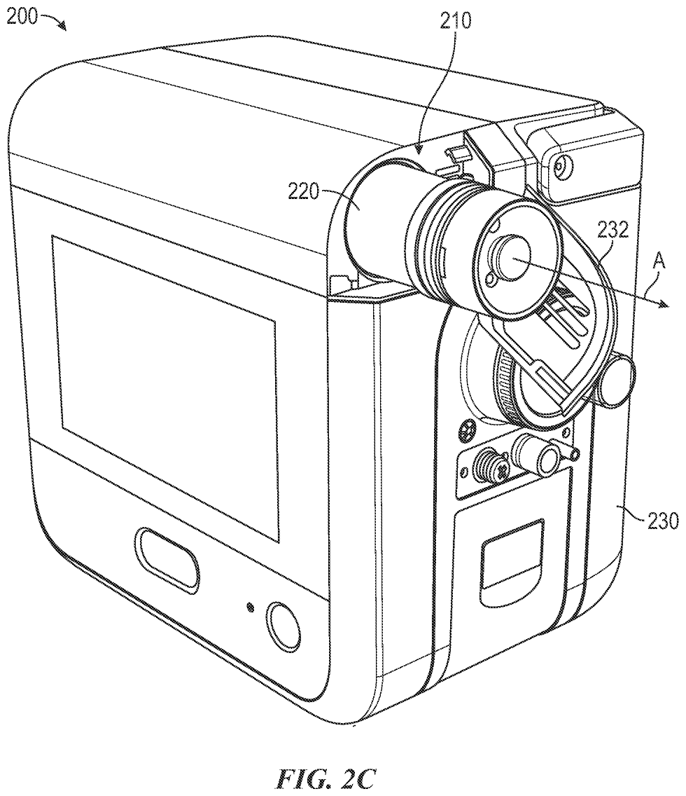

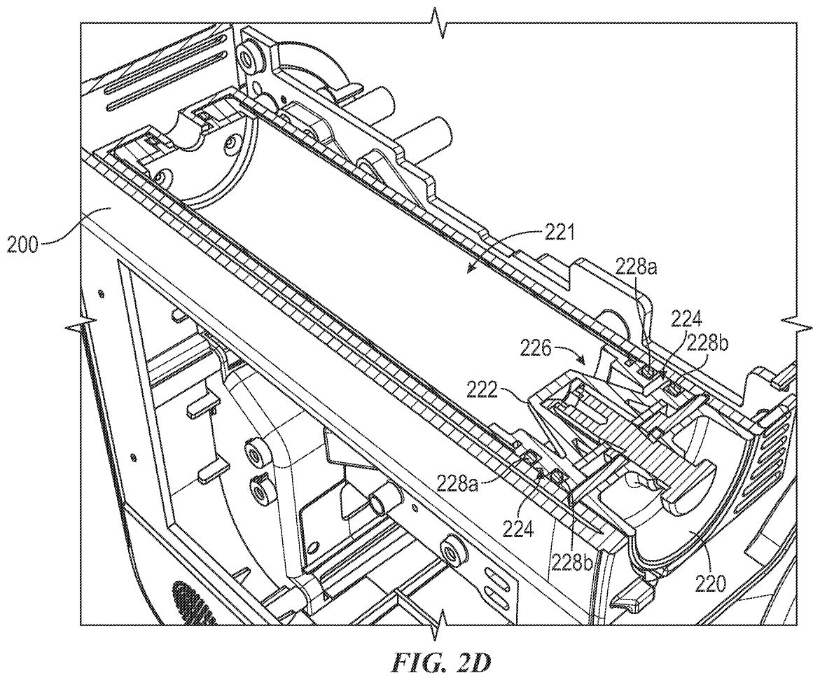

As set forth above, the ventilators described herein can include a removable and replaceable adsorption bed for producing concentrated oxygen from ambient air. FIGS. 2A-2C, for example, are a series of perspective views illustrating a ventilator 200 having a removeable adsorption bed 220 and configured in accordance with select embodiments of the present technology. Referring to FIG. 2A, the ventilator 200 can have a housing 230, a main ventilator connection 206 (which can be the same or similar to the main ventilator connection 106 described with respect to FIG. 1), an oxygen outlet port 208 (which can be the same or similar to the oxygen outlet port 108 described with respect to FIG. 1), and a breath sensing port 214 (which can be the same or similar to the breath sensing port 114 described with respect to FIG. 1).

The housing 230 can include an access door or panel 232 that can be selectively moved between a closed position shown in FIG. 2A and an open position shown in FIG. 2B. The access door 232 can be coupled to the housing 230 via any suitable arrangement that permits the access door 232 to be repeatedly moved between the open position and the closed position. For example, as shown in FIG. 2B, the access door 232 can be coupled to the housing 230 via coupling member 234 (e.g., a tether) that keeps the access door 232 coupled to the housing when in the open position. In other embodiments, the access door 232 can be coupled to the housing 230 via a hinge, which can optionally be biased to keep the access door 232 in the closed position. In some embodiments, the access door 232 can be releasably secured to the housing 230 in the closed position via a friction fit, a snap fit, a magnetic connection, or other suitable connection known in the art.

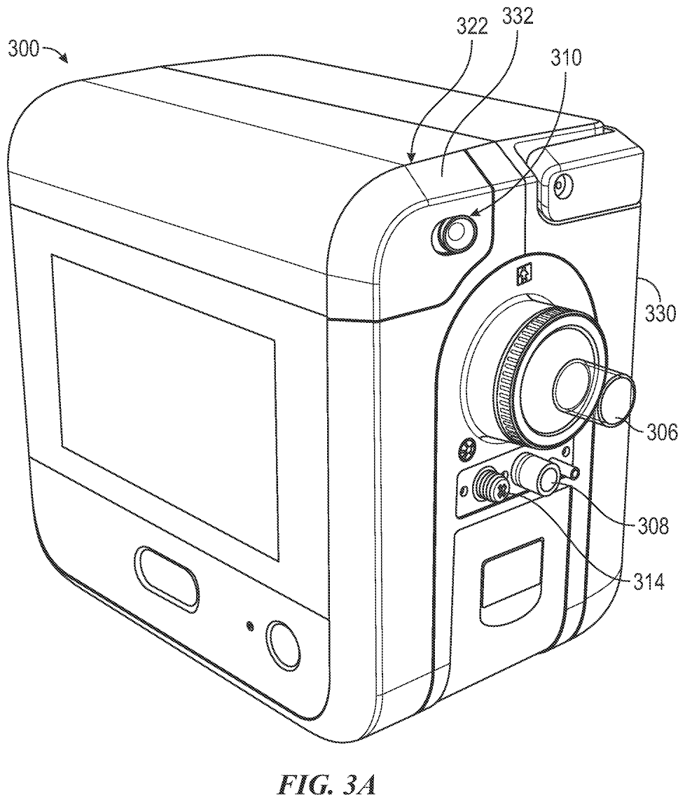

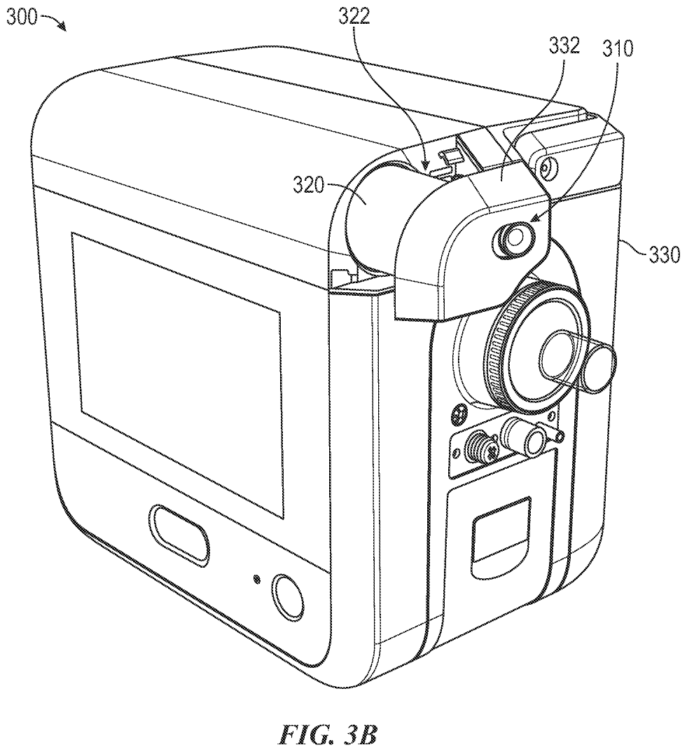

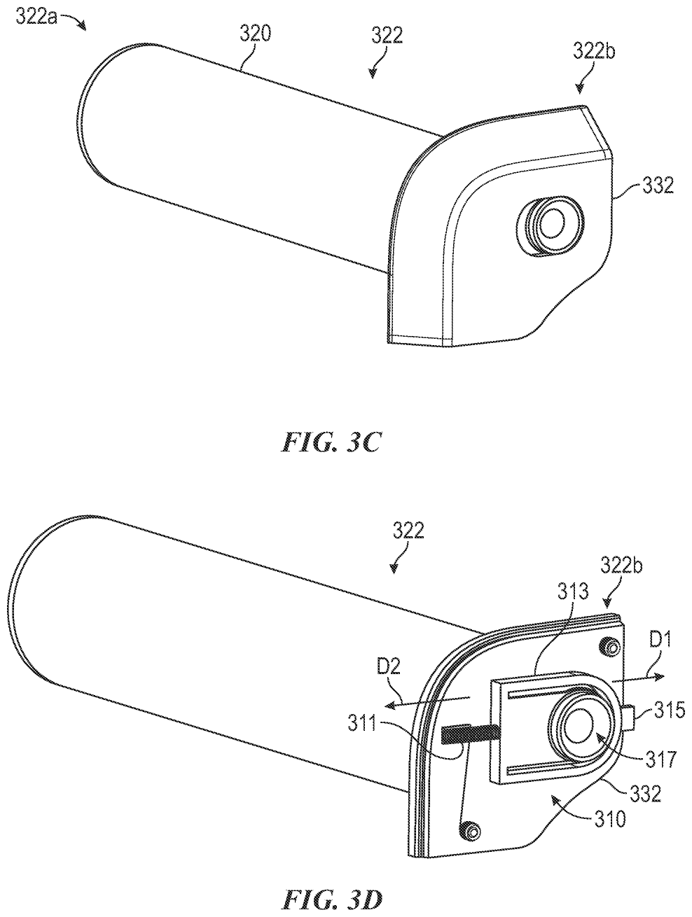

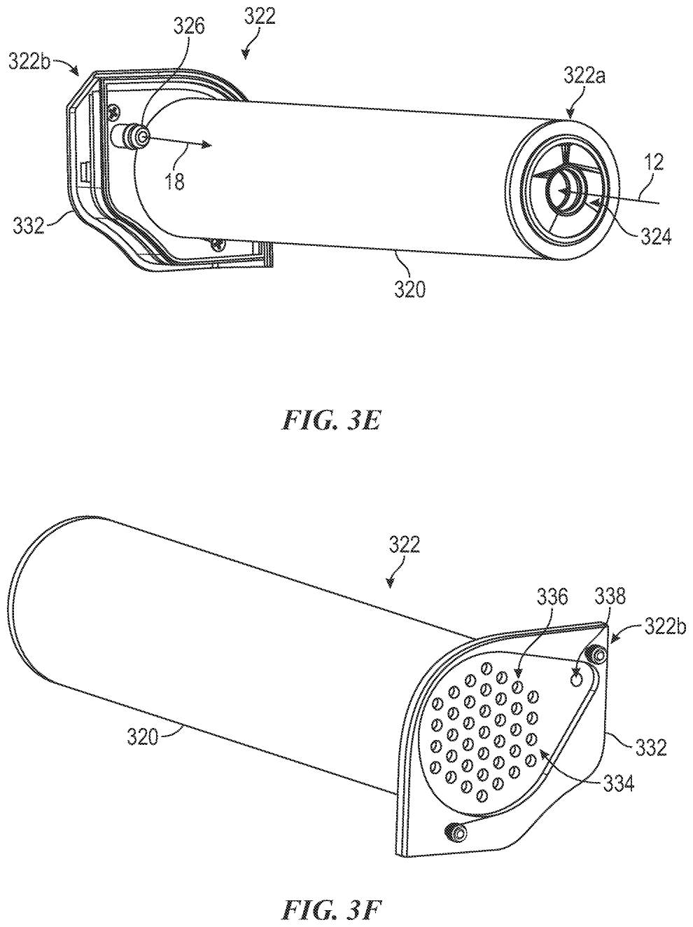

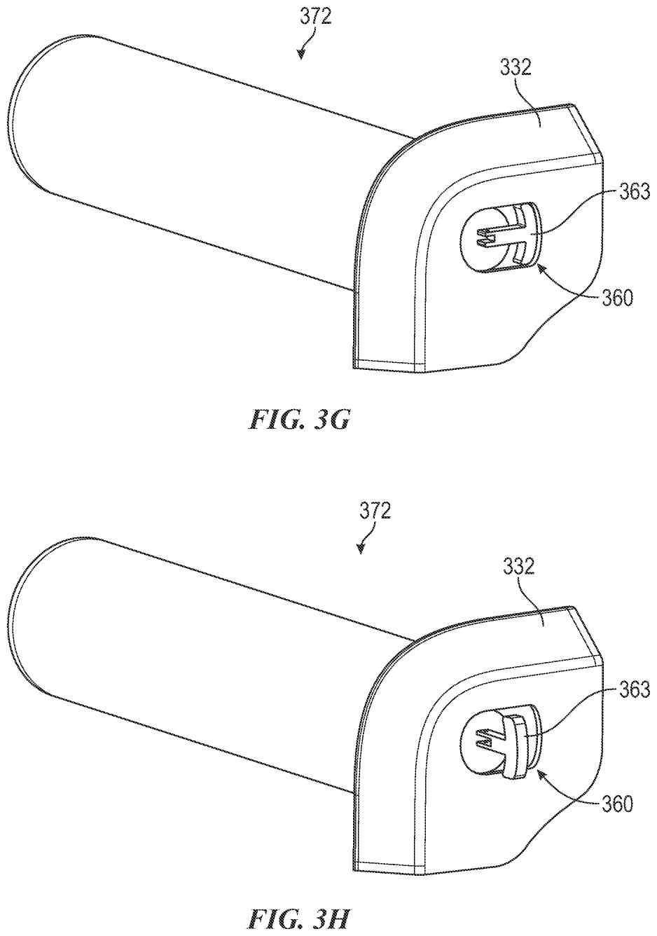

The access door 232 can be configured to provide access to at least a portion of an oxygen assembly 210 positioned within an interior of the ventilator 200 and configured to generate concentrated oxygen. In particular, the access door 232 can be aligned with an adsorption bed 220 of the oxygen assembly 210. For example, as shown in FIGS. 2B and 2C, when the access door 232 is in the open position, a user (not shown) can partially or fully remove the adsorption bed 220 from the oxygen assembly 210, e.g., by moving the adsorption bed 220 in the direction indicated by arrow A in FIG. 2C. In some embodiments, the adsorption bed 220 can be removed from the ventilator 200 without the use of any tools (e.g., the user can fully remove the adsorption bed 220 simply using their hands). In other embodiments, one or more readily available tools (e.g., an Allen wrench, a Philips-head screwdriver, etc.) may be required for the user to remove the adsorption bed 220 from the ventilator 200, such as for the embodiment described below with reference to FIGS. 3I and 3J. Once the user has removed the adsorption bed 220 from the ventilator 200, the user can insert a new adsorption bed (not shown) into the ventilator 200 by sliding the new adsorption bed into the same slot previously occupied by the adsorption bed 220. In some embodiments, the new adsorption bed can be installed in the ventilator 200 without the use of any tools (e.g., the user can fully install the new adsorption bed simply using their hands). In other embodiments, one or more readily available tools (e.g., an Allen wrench, a Philips-head screwdriver, etc.) may be required for the user to install the new adsorption bed in the ventilator 200. The user can then move the access door 232 to the closed position (FIG. 2A) to retain the new adsorption bed within the housing 230. As best shown in FIG. 2A, in some embodiments the access door 232 may further include one or more vents or exhaust openings 212 (which can be the same or similar to the outlet vent 112 described with respect to FIG. 1) such that the oxygen assembly 210 can purge byproduct gases (e.g., nitrogen rich gas) to an environment external to the ventilator 200.

FIG. 2D is a perspective, cross-sectional view of the adsorption bed 220 positioned within the ventilator 200. As shown in FIG. 2D, the adsorption bed 220 can include a seal assembly 222 having an annular fluid channel 224 (“the annular channel 224”) extending at least partially or fully around a perimeter (e.g., circumference) of the seal assembly 222. In some embodiments, the annular channel 224 is formed partially or fully between a first annular ridge or sealing element 228a (e.g., a first O-ring, a first lip seal, etc.) and a second annular ridge or sealing element 228b (e.g., a second O-ring, a second lip seal, etc.) (referred to collectively as the sealing elements 228), both of which may also extend at least partially or fully around a perimeter (e.g., circumference) of the seal assembly 222. The sealing elements 228 can be configured to form a substantially air-tight seal with an internal portion or surface of the ventilator 200 configured to receive the adsorption bed 220 such that, when the adsorption bed 220 is inserted into the ventilator 200, the annular channel 224 forms a toroid-shaped chamber extending around an exterior circumference of the adsorption bed 220. In some embodiments, the annular channel 224 and sealing elements 228 are generally circular, although in other embodiments the annular channel 224 and the sealing elements 228 can be triangular, square, or any other suitable shape that generally corresponds to the shape formed by the internal surface of the ventilator 200 configured to receive the adsorption bed 220. Additionally, the annular channel 224 and the sealing elements 228 can be shaped to correspond to a cross-sectional shape of the adsorption bed 220.

The annular channel 224 can be in fluidic communication with an interior 221 of the adsorption bed 220 via one or more flow paths 226 extending at least partially through the seal assembly 222. Although only one flow path 226 is shown in the FIG. 2D, in other embodiments the seal assembly 222 can include two, three, four, five, or any other suitable number of flow paths 226. When inserted into the ventilator 200, the annular channel 224 can also be in fluidic communication with one or more ports (not shown) in the internal surface of the ventilator 200 that receives the adsorption bed. The ports can be fluidically coupled to an oxygen chamber (e.g., the oxygen chamber 121 shown in FIG. 1) or an oxygen outlet port (e.g., the oxygen outlet port 108 shown in FIG. 1). Accordingly, oxygen generated within the interior 221 of the adsorption bed 220 can flow through the flow paths 226 into the annular channel 224, and from the annular channel 224 into a downstream portion of the oxygen assembly 210 and/or ventilator 200 for delivery to a patient.

Adsorption beds including annular channels such as the annular channel 224 are expected to exhibit several advantages. For example, the adsorption bed 220 can be easier to correctly position within the ventilator 200. Many conventional adsorption beds must be inserted into associated oxygen assemblies in a specific orientation (e.g., with the adsorption bed at a specific radial orientation to the ventilator) and/or with a tapered portion of the adsorption bed positioned to be received by a correspondingly tapered portion of the associated oxygen concentrator, e.g., to insure proper alignment between air flow ports of the oxygen assembly and the adsorption bed. In contrast, the annular channel 224 of the adsorption bed 220 can extend around the entire perimeter of the seal assembly 222 such that the adsorption bed 220 can be inserted into the ventilator 200 in any radial orientation while still forming the fluidic connections that enable gases to flow through the adsorption bed (e.g., the flow paths 226 will be fluidically coupled to the one or more ports (not shown) in the internal surface of the ventilator 200 regardless of the radial orientation the adsorption bed 220 inserted in). Additionally, because the annular channel 224 can be fluidly coupled to the interior 221 of the adsorption bed 220 via one or more fluid paths 226, one or more dimensions (e.g., length, width, cross-sectional area, etc.) of individual ones of the fluid paths 226 can be reduced which, in turn, can increase the pressure integrity of the adsorption bed 220 compared to other conventional adsorption beds.

FIGS. 3A and 3B illustrate another ventilator 300 having a removeable adsorption bed 320 and configured in accordance with embodiments of the present technology. The ventilator 300 can be generally similar to the ventilator 100 (FIG. 1) and/or the ventilator 200 (FIGS. 2A-2C) However, relative to the ventilator 200, which has an access door 232 forming part of the ventilator housing 230, the ventilator 300 includes an adsorption bed 320 (FIG. 3B) having an integrated panel 332 that forms part of a housing 330 of the ventilator 300 (the adsorption bed 320 and the integrated panel 332 collectively form an “adsorption bed assembly 322” or simply “oxygen assembly 322”). As shown in FIG. 3A, the integrated panel 332 aligns with an external surface of the housing 330. The integrated panel 332 can include an attachment or latching mechanism 310 operable to releasably couple the integrated panel 332 (and thus the adsorption bed 320) to the housing 330. As described in greater detail with respect to FIG. 3D, the attachment mechanism 310 can be actuated by a user to decouple the integrated panel 332 from the housing 330, such that the adsorption bed assembly 322 can be removed from the ventilator 300 (e.g., as shown in FIG. 3B). Without being bound by theory, integrating the panel 332 with the adsorption bed 320 is expected to reduce the volume within the ventilator 300 that the panel 332 occupies, which in turn enables a larger adsorption bed 320 to be utilized. This is expected to be advantageous because, as a skilled artisan will recognize, a relatively larger adsorption bed generally has a longer useable life, and therefore enables a user to generate concentrated oxygen for a longer period of time before having to replace the adsorption bed assembly 322.

FIG. 3C is a front perspective view of the adsorption bed assembly 322 removed from the ventilator 300. As shown, the adsorption bed assembly 322 can have a first end portion 322a and a second end portion 322b opposite the first end portion 322a. The second end portion 322b can include all or part of the integrated panel 332.

FIG. 3D is another perspective view of the adsorption bed assembly 322 with select aspects of the integrated panel 332 omitted to better show additional features of the attachment mechanism 310. For example, in the illustrated embodiment, the attachment mechanism 310 includes a biasing element 311 operably coupled to a latching member 313. In the illustrated embodiment, the biasing element 311 is a compression spring configured to bias the latching member 313 in a first direction D1. In other embodiments, the biasing element 311 can include any other suitable biasing element, and/or can be configured to bias the latching member 313 in any other suitable direction. The latching member 313 can include a protrusion 315 configured to be releasably received by a retention feature (not shown) of the housing 330 of the ventilator 300 (FIGS. 3A and 3B). The latching member 313 can be slidably coupled to the adsorption bed assembly 322 such that it can move relative to the integrated panel 332 in the first direction D1 and a second direction D2 opposite the first direction D1. In some embodiments, the latching member 313 can further include a switch or actuator 317 to allow a user to operate the attachment mechanism 310. In operation, the biasing element 311 can exert a force on the latching member 313 in the first direction D1, e.g., to bias the latching member 313 in the first direction D1 toward the ventilator housing 330 (FIGS. 3A and 3B), such that the protrusion 315 is releasably received by the retention feature of the housing 330. With the biasing element 311 in this configuration, the adsorption bed assembly 322 can be retained within the ventilator 300. To remove the adsorption bed assembly 322 from the ventilator 300, the latching member 313 can be moved in the second direction D2 by a user sliding the actuator 317 in the second direction D2, e.g., to oppose the force exerted on the latching member 313 by the biasing element 311. As the latching member 313 moves in the second direction D2, the protrusion 315 can be released from/disengage the retention feature of the ventilator housing 330, which, in turn, can allow the adsorption bed assembly 322 to be removed from the ventilator 300, as described previously regarding FIGS. 3A and 3B. In some embodiments, the latching member 313 must be pressed in (e.g., in a direction parallel to a longituindal axis of the adsorption bed 320) before it can slide in the second direction D2, to provide an extra level of security to reduce the risk of accidental dislodgement or removal of the adsorption bed assembly 322. Although FIG. 3D illustrates use of a biasing element 311 and a protrusion 315, other suitable user-operable attachment mechanisms can be used to removably secure the adsorption bed assembly 322 to the ventilator 300.

The adsorption bed assembly 322 is configured to be fluidically coupled to other portions of the ventilator 300 when inserted into the ventilator 300 to complete a pathway through an oxygen assembly (not shown) within the ventilator 300. For example, FIG. 3E is a rear perspective view of the adsorption bed assembly 322 illustrating air flow ports for fluidly connecting the adsorption bed assembly 322 to the ventilator 300. In some embodiments, the first end portion 322a can include a first aperture or port 324 that aligns with an air intake of the oxygen assembly or ventilator 300 (e.g., the patient air intake 104 shown in FIG. 1). Accordingly, the first port 324 can place an interior of the adsorption bed 320 in fluidic communication with the air intake, such that ambient air (e.g., the air 12) can flow into the adsorption bed 320 via the first port 324. Additionally, the second end portion 322b can include a second aperture or port 326 also in fluid communication with an interior of the adsorption bed 320, such that concentrated oxygen 18 can flow out of the adsorption bed 320 via the second port 326. The second port 326 can be configured to align with or otherwise be fluidly coupled to an oxygen chamber (e.g., the oxygen chamber 121 shown in FIG. 1) or other oxygen outlet (e.g., the oxygen outlet port 108 shown in FIG. 1). Accordingly, gases can flow through the adsorption bed 320 from the first port 324 to the second port 326 (e.g., during the adsorption phase). In other embodiments, the direction of gas flow can be reversed, such that gases flow through the adsorption bed 320 from the second port 326 toward the first port 324 (e.g., during the desorption phase). In the illustrated embodiment, the first port 324 and the second port 326 are generally parallel, e.g., configured to direct the flow of gases (e.g., air 12, concentrated oxygen 18, etc.) along axes that are generally parallel to each other. In other embodiments, the port 324 and the port 326 can have any other suitable orientation relative to each other, such as perpendicular (e.g., as illustrated for the in FIG. 2D) or angled.

FIG. 3F is yet another front perspective view of the adsorption bed assembly 322, with select aspects of the integrated panel 332 and the attachment mechanism 310 omitted to better show the flow path through the adsorption bed assembly 322. In particular, the integrated panel 332 can include a recessed or fluid transfer area 334. The fluid transfer area 334 can be in fluidic communication with an interior of the adsorption bed 320 via one or more vents or openings 336 within the fluid transfer area 334. The fluid transfer area 334 can further include an aperture 338 fluidly coupled to the second port 326 (FIG. 3E). Accordingly, in at least some embodiments, fluid (e.g., oxygen) can exit the adsorption bed 320 via one or more of the vents 336, pass through the fluid transfer area 334, enter the aperture 338, and exit the adsorption bed assembly 322 via the second port 326 (the direction of flow is reversed during the desorption phase). This fluid transfer can occur at least partially within the integrated panel 332, which can increase an effective length of the adsorption bed 320. For example, reducing (e.g., minimizing) the volume of the adsorption bed 320 used for fluid transfer within the ventilator 300 can correspondingly increase the volume within the interior (not shown) of the adsorption bed 320, such that the adsorption bed 320 can carry an increased amount of nitrogen-adsorbent material compared to other adsorption beds. Although not shown in FIG. 3F, the second end portion 332b can include one or more plates or sealing members configured to form a substantially air-tight seal with the fluid transfer area 334, such that substantially all the fluid (e.g., oxygen) generated within the adsorption bed 320 can exit the adsorption bed assembly 322 via the aperture 338 and the associated second port 326.

FIGS. 3G and 3H are perspective views of another adsorption bed assembly 372 configured in accordance with embodiments of the present technology. The adsorption bed assembly 372 can be generally similar to the adsorption bed assembly 322 of FIGS. 3A-3F, and can be configured for use with the ventilator 300. However, the adsorption bed assembly 372 includes an attachment mechanism 360 having a latching member 363 that is pivotally coupled to the integrated panel 332. In the illustrated embodiment, the latching member 363 is generally T-shaped, although the latching member 363 can have other shapes. In operation, the latching member 363 can be pivoted from a first position (FIG. 3G) relative to the integrated panel 332 to and/or toward a second position (FIG. 3H) relative to the integrated panel 332, e.g., to allow the adsorption bed assembly 372 to be removed from the ventilator 300 (FIGS. 3A and 3B) as described previously. In some embodiments, transitioning the latching member 363 from the first position to the second position unlocks or releases a flange, tongue, or other retention element (not shown) securing the adsorption bed assembly 372 to the housing 330 of the ventilator 300. In some embodiments, in the second position, the latching member 363 can serve as a handle, e.g., to aid a user in removing the adsorption bed assembly 372 from the ventilator 300. In some embodiments, the latching member 363 must be rotated (e.g., in clockwise or counterclockwise direction) after it has been moved to the second position to disengage the flange, tongue, or other retention element (not shown) to provide an extra level of security to reduce the risk of accidental dislodgement or removal of the adsorption bed assembly 372. In the illustrated embodiment, the latching member 363 is generally perpendicular to a longitudinal axis of the adsorption bed assembly 372 in the first position and generally parallel to the longitudinal axis of the adsorption bed assembly 372 in the second position. In other embodiments, the latching member 363 can have any other suitable orientation relative to the adsorption bed assembly 372 in the first and second positions.

FIGS. 3I and 3J are perspective views of another adsorption bed assembly 382 configured in accordance with embodiments of the present technology. The adsorption bed assembly 382 can be generally similar to the adsorption bed assembly 322 of FIGS. 3A-3F and the adsorption bed assembly 372 of FIGS. 3G and 3H, and therefore can be configured for use with the ventilator 300. However, the adsorption bed assembly 382 includes a locking/unlocking or attachment mechanism 381 including a wire bail 386 and a keyhole 388, and an elastomeric cover 384 for covering the attachment mechanism 381 (the cover 384 is shown removed from, and thus providing access to, the attachment mechanism 381 in FIGS. 3I and 3J for purposes of illustration). Both the keyhole 388 and the wire bail 386 are moveable between respective locked positions (FIG. 3I) and unlocked positions (FIG. 3J). In some embodiments, both the keyhole 388 and the wire bail 386 must be in their respective unlocked positions to remove the adsorption bed assembly 382 from a ventilator (e.g., the ventilator 300). For example, in operation, a user can remove the adsorption bed assembly 382 from a ventilator/ventilator housing by removing the cover 384 to access the attachment mechanism 381. With the attachment mechanism 381 exposed, a user can insert a tool (e.g., a flat-head screw driver, a hex-head screwdriver, etc.; not shown) into the keyhole 388 and rotate it approximately 90 degrees counterclockwise to the configuration shown in FIG. 3J to at least partially unlock or disengage an internal latching mechanism (not shown) mechanically holding the adsorption bed assembly 382 to the ventilator 300. To fully unlock or disengage the internal latching mechanism, a user can then flip the wire bail 386 to the configuration shown in FIG. 3J. A user can then use the wire bail 386 to pull the adsorption bed assembly 382 out of the ventilator housing (not shown). Once the adsorption bed assembly 382 is extracted from the ventilator housing, a replacement cartridge can be installed by performing the above recited procedure in reverse.

Although the embodiments shown in FIGS. 1-3J are described in the context of ventilator systems that include an integrated oxygen concentrator or oxygen assembly, one skilled in the art will appreciate that certain features described with respect to FIGS. 1-3J can be implemented in oxygen concentrators that are not part of a ventilator system. For example, the oxygen assembly 110 or components thereof (FIG. 1), the oxygen assembly 210 or components thereof (FIGS. 2A-2D), and/or the adsorption bed assembly 322 or components thereof (FIGS. 3A-3J) can be integrated into a standalone oxygen concentrator that does not also provide ventilation therapy. Accordingly, the present technology also includes oxygen concentrators having removable adsorption beds and oxygen assemblies that can be the same as, or generally similar to, the corresponding removable adsorption beds and oxygen assemblies described above.

### II. Additional Features of Oxygen Assemblies Having Adsorption Beds

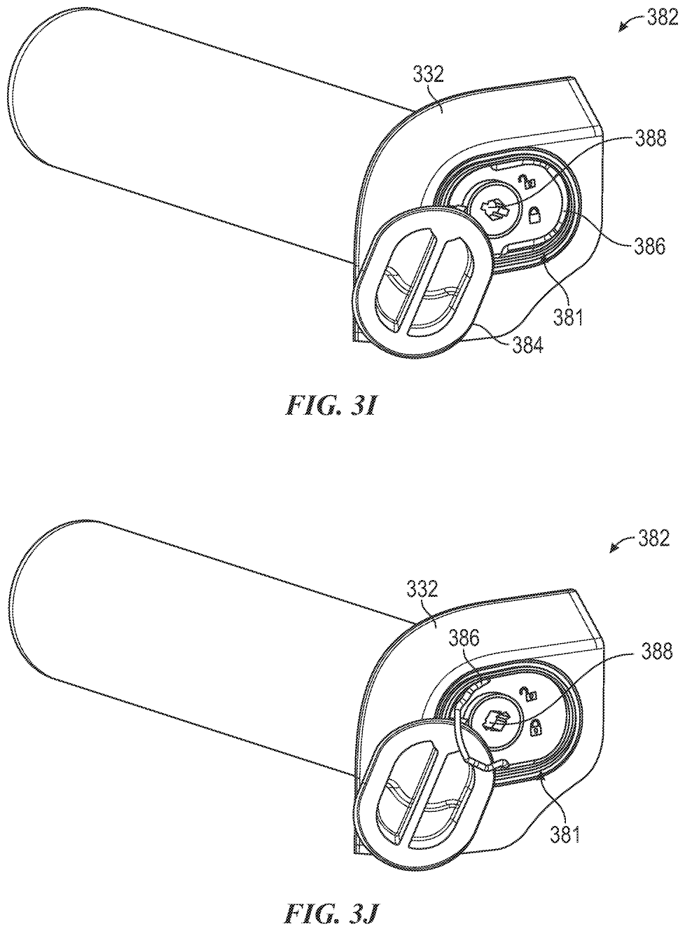

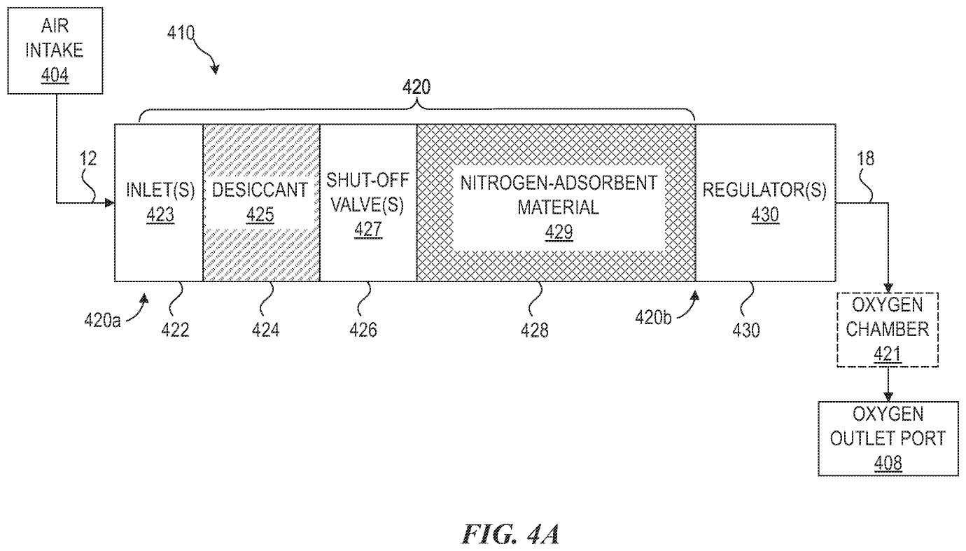

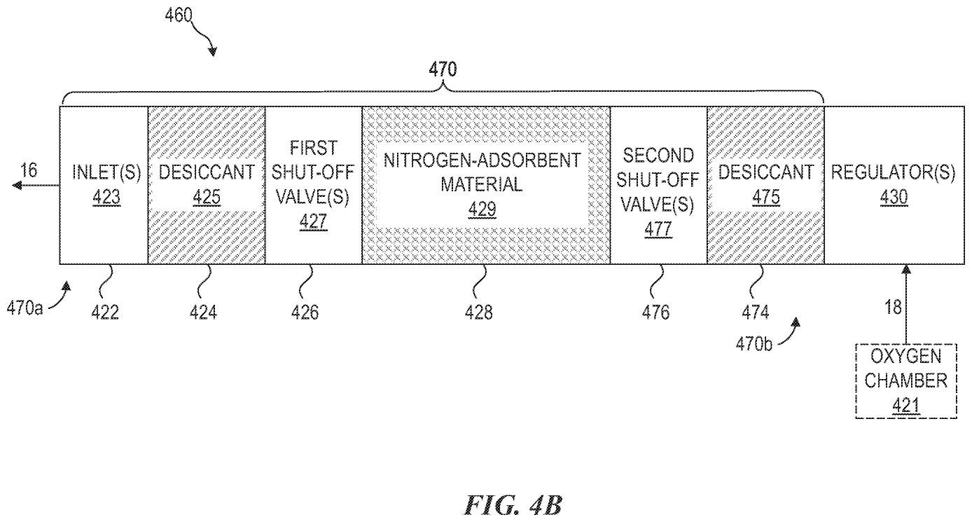

FIG. 4A is a schematic illustration of an oxygen assembly 410 including an adsorption bed 420 and configured in accordance with embodiments of the present technology. The oxygen assembly 410 and the adsorption bed 420 can be generally similar to or the same as the oxygen assemblies and adsorption beds described with reference to FIGS. 1-3J, and can therefore be used with the ventilators 100-300 previously described, other ventilators with integrated oxygen production, and/or standalone oxygen concentrators. The adsorption bed 420 can include a first end 420a and a second end 420b opposite the first end 420a. The adsorption bed 420 can further include a fluid inlet portion 422, a moisture-capturing or desiccant material portion 424 for capturing and retaining moisture or humidity in air entering the adsorption bed 420 via the inlet portion 422, a shut-off valve portion 426, and a nitrogen-adsorbent material portion 428 for capturing and retaining nitrogen from the de-humidified air. In the illustrated embodiment, the inlet portion 422 is positioned proximate the first end 420a of the adsorption bed 420, the desiccant material portion 424 is positioned between the inlet portion 422 and the shut-off valve portion 426, the shut-off valve portion 426 is positioned between the desiccant material portion 424 and the nitrogen-adsorbent material portion 428, and the nitrogen-adsorbent material portion 428 is positioned between the shut-off valve portion 426 and the second end 420b.

The fluid inlet portion 422 can include one or more fluid inlets 423, and individual ones of the fluid inlets 423 can be fluidly coupled to an air intake 404 (e.g., the patient air intake 104 shown in FIG. 1) and configured to selectively and/or independently allow air 12 received via the patient air intake 404 to flow into the adsorption bed 420. The desiccant material portion 424 can include one or more moisture-capturing or desiccant materials 425 (e.g., alumina) configured to preferentially capture moisture (e.g., water) from the air 12 when the air 12 passes through the desiccant material portion 424, thereby at least partially de-humidifying the air 12 before it flows into the nitrogen-adsorbent material portion 428. The shut-off valve portion 426 can include one or more shut-off valves 427, and individual ones of the shut-off valves 427 can be selectively and/or independently transitioned between (i) a first (e.g., open) configuration in which the shut-off valves 427 permit air to flow through the shut-off valve portion 426 between the desiccant material portion 424 and the nitrogen-adsorbent material portion 428, and (ii) a second (e.g., closed) configuration in which the shut-off valves 427 at least partially or fully prevent the air 12 from flowing through the shut-off valve portion 426 between the desiccant material portion 424 and the nitrogen-adsorbent material portion 428. The nitrogen-adsorbent material portion 428 can include one or more nitrogen-adsorbent materials 429 (e.g., zeolite) configured to preferentially adsorb nitrogen from the air 12 when the air 12 passes through the nitrogen-adsorbent material portion 428, e.g., to generate concentrated oxygen 18, which can then flow into an oxygen chamber 421 and/or directly to an oxygen outlet port 408.

In some embodiments, the oxygen assembly 410 may further include one or more regulators 430 fluidly coupled to the second end 420b of the adsorption bed 420. Individual ones of the regulators 430 can be configured to selectively and/or independently control the flow of fluid (e.g., oxygen 18) through the second end 420b of the adsorption bed 420. Additionally, one or more of the regulators 430 can be fluidly coupled to the oxygen chamber 421 and/or the oxygen outlet port 408. The oxygen assembly 410 may also include one or more air flow valves (not shown) positioned between the air intake 404 and the inlets 423. The air flow valves can be selectively actuated to permit gas to flow between the air intake 404 and the inlets 423 (e.g., during use of the oxygen assembly 410) and to block gas flow between the air intake and the inlets 423 (e.g., when the oxygen assembly is not in use). In some embodiments, the air flow valves can be coupled to the adsorption bed 420, although in other embodiments the air flow valves are separate from the adsorption bed 420.

In operation, the adsorption bed 420 can receive air 12 from the patient air intake 404 via one or more of the inlets 423. The air 12 can flow through the inlet portion 422 and enter the desiccant material portion 424, in which the desiccant material 425 can preferentially capture and retain moisture from the air 12, e.g., to dehumidify the air 12. When one or more of the shut-off valves 427 are in the first (e.g., open) configuration, the dehumidified air can flow from the desiccant material portion 424, through the shut-off valve(s) 427, and enter the nitrogen-adsorbent material portion 428. When the dehumidified air is in the nitrogen-adsorbent material portion 428, the nitrogen-adsorbent material 429 can preferentially capture and retain nitrogen from the air 12. The adsorption of nitrogen from the air 12 in the nitrogen-adsorbent material portion 428 can generate concentrated oxygen 18, which can flow through one or more of the regulators 430 and toward the oxygen outlet port 408, e.g., for delivery to a patient. When the adsorption bed 420 is no longer in use, all or a subset of the shut-off valves 427 can be transitioned to and/or toward the second configuration to fluidly isolate the desiccant material portion 424 from the nitrogen-adsorbent material portion 428.

Some nitrogen-adsorbent materials, such as zeolite, can degrade if exposed to moisture. To reduce this degradation, some adsorption beds include a desiccant material positioned immediately upstream of the nitrogen-adsorbent material to at least partially prevent moisture from reaching the nitrogen-adsorbent material as air flows through the adsorption bed. However, when air is not flowing through the adsorption beds (e.g., when the oxygen assembly is turned off or in storage), some of the moisture in the desiccant material can migrate into the nitrogen-adsorbent material, expediting the degradation of the nitrogen-adsorbent material therein. Because the desiccant material is positioned within the same housing as the nitrogen-adsorbent material, this moisture migration can occur even if the adsorption bed is fluidly sealed off from the external environment during storage periods. In contrast, the shut-off valve portion 426 of the adsorption bed 420 includes one or more shut-off valves 427 positioned between the desiccant material portion 424 and the nitrogen-adsorbent material portion 428, and individual ones of the one or more shut-off valves 427 can be closed to reduce and/or prevent moisture captured by the desiccant material 425 from migrating to and degrading the nitrogen adsorbent material 429. For example, when the adsorption bed 420 is not in use, one or more (e.g., all) the shut-off valves 427 can be transitioned from the first (e.g., open) configuration to and/or toward the second (e.g., closed) configuration, such that the shut-off valves 427 at least partially or fully separate (e.g., fluidly isolate) the desiccant material 425 (and any moisture captured by the desiccant material 425) from the nitrogen-adsorbent material 429. Accordingly, adsorption beds configured in accordance with embodiments of the present technology are expected to reduce moisture transfer between the desiccant material and the nitrogen-adsorbent material, which can reduce the degradation of the nitrogen-adsorbent material and/or improve the operational lifetime of these adsorption beds.

The adsorption bed 420 prevents or at least reduces moisture from entering the nitrogen adsorbent material 429 during the adsorption phase of the oxygen generation process. In some embodiments, a desiccant material can also be positioned between the nitrogen-adsorbent material 429 and the regulator 430 to prevent or at least reduce moisture in gases used during the desorption phase of the oxygen generation process from entering the nitrogen-adsorbent material. For example, FIG. 4B illustrates an oxygen assembly 460 having an adsorption bed 470 and configured in accordance with select embodiments of the present technology. The oxygen assembly 460 can be generally similar to the oxygen assembly 410 (FIG. 4A). For example, the adsorption bed 470 can have a first end 470a, a second end opposite the first end 470b, the first desiccant material portion 424 having the first desiccant material 425, the first shut-off valve portion 426 having the first shut-off valve 427, and the nitrogen-adsorbent material portion 428 having the nitrogen-adsorbent material 429. However, relative to the oxygen assembly 410, the oxygen assembly 460 further includes a second desiccant material portion 474 containing a desiccant material 475 positioned between the nitrogen adsorbent portion 428 the second end 470b. The desiccant material 475 can be the same as the desiccant material 425, or can be a combination of a nitrogen-adsorbent material and a desiccant material. During the desorption phase, gas (e.g., the oxygen 18) flows through the adsorption bed 420 from the second end 470b toward the first end 470a to regenerate the nitrogen-adsorbent material 429. Nitrogen rich gas 16 flows out of the inlets 423 and is purged to the external environment via one or more vents (not shown). However, especially after periods of non-use, the oxygen 18 in the oxygen chamber 421 may have increased moisture content (e.g., due to leaks in the oxygen chamber 421). Accordingly, the desiccant material 475 in the second desiccant material portion 474 captures and retains any moisture in oxygen 18 used during the desorption phase before the oxygen 18 enters the nitrogen-adsorbent material 429.

In addition to or in lieu of having the second desiccant material portion 474, the oxygen assembly 460 and/or the oxygen chamber 421 can be composed of materials that have a relatively low moisture vapor transmission rate (“MVTR”) to further minimize or at least reduce unwanted moisture ingress into the system, thereby minimizing or at least reducing moisture that may be pushed back through the adsorption bed 470 during the desorption phase. For example, the oxygen assembly 460, the oxygen chamber 421, any conduits or flow paths connecting the oxygen assembly 460 and the oxygen chamber 421, and/or any other intervening structures can be composed of materials that permit little to no water vapor to leak/permeate therethrough. As a result, the oxygen stored in the oxygen chamber 421 and used for the desorption phase is expected to retain a low moisture content even during prolonged periods of storage, and regardless of the humidity of the environment in which the system is stored or used. Suitable materials having a relatively high MVTR include, but are not limited to metals (e.g., aluminum, titanium, copper, nickel, etc.,) alloys (e.g., stainless steel, brass, bronze, etc.), and/or metal plating. For optically transparent components, a relatively high MVTR clear plastic such as polyethylene terephthalate (PET), polyethylene terephthalate glycol (PETG), or cyclic olefin copolymer (COC) can be used. For o-rings or other rubber components, a relatively high MVTR rubber material such as ethylene propylene diene monomer (EPDM)) can be used. Additional materials can include polymers such as polyethyleneimine (PEI) and/or high-density polyethylene (HDPE). In some embodiments, the material has an MVTR value of 8 g/m² or less, such as 6 g/m² or less, 5 g/m² or less, 4.5 g/m² or less, 4 g/m² or less, 3.5 g/m² or less, or 3 g/m² or less.

The adsorption bed 470 can further include a second shut-off valve portion 476 having a second shut-off valve 477 positioned between the nitrogen adsorbent material portion 428 and the second desiccant material portion 474. The second shut-off valve 477 can be generally similar to the first shut-off valve 427 described in detail with respect to FIG. 4A, except that the second shut-off valve 477 prevents or at least reduces moisture captured in the second desiccant material portion 474 from migrating into the nitrogen-adsorbent material portion when the oxygen assembly 460 is not being used.

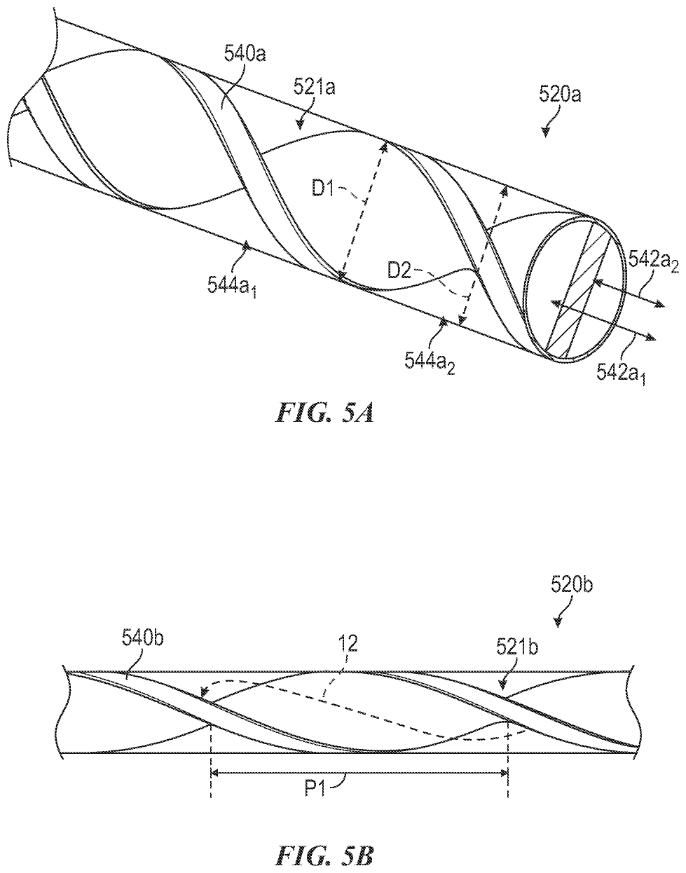

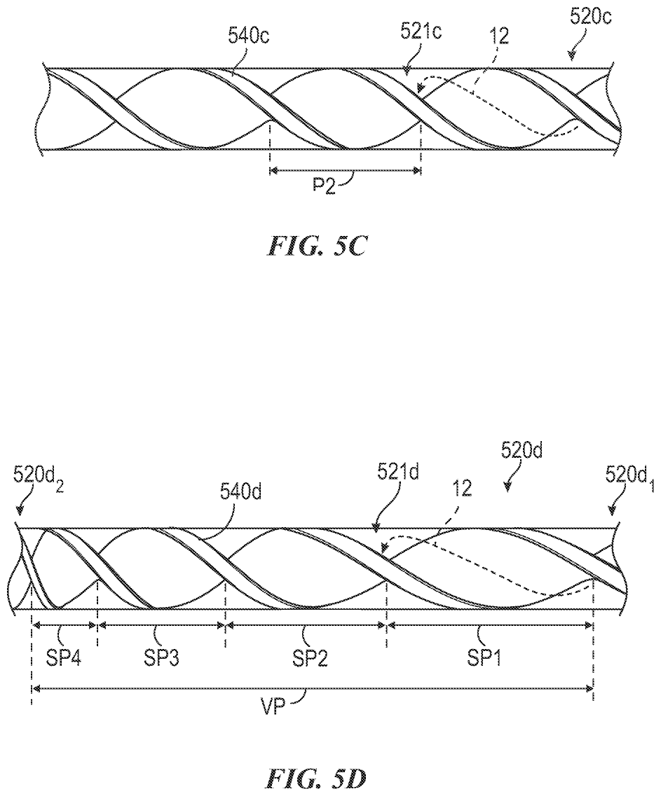

The present technology also includes adsorption beds having improved aspect ratios. For example, FIGS. 5A-5D illustrate respective adsorption beds 520a-d having associated spiraled or helical inserts 540a-d configured in accordance with embodiments of the present technology. Specifically, FIG. 5A is a perspective view of an adsorption bed 520a including a helical insert 540a, FIG. 5B is a side view of an adsorption bed 520b including a helical insert 540b with a relatively low pitch, FIG. 5C is a side view of an adsorption bed 520c including a helical insert 540c with a relatively high pitch, and FIG. 5D is a side view of an adsorption bed 520d having a helical insert 540d with a longitudinally-varied pitch. The following descriptions of FIGS. 5A-5D reference select aspects of each of the respective adsorption beds 520a-d and the associated helical inserts 540a-d. Although these aspects are described with reference to a specific adsorption bed and/or a specific helical insert for the sake of illustrative clarity, it will be appreciated that any description of one helical insert (e.g., the helical insert 540a of FIG. 5A) can apply equally to one or more of the other helical inserts of the present technology (e.g., one or more of the helical inserts 540b-d of FIGS. 5B-D), and vice versa.

Each of the helical inserts 540a-d can be positioned within a corresponding interior 521a-d of the respective adsorption beds 520a-d and extend in a direction generally parallel to a longitudinal axis of the respective adsorption bed 520a-d, e.g., at least partially or fully along a length of the respective interior 521a-d. As described previously, the interior 521a-d of each of the adsorption beds 520a-d can be filed with a nitrogen-adsorbent material (not shown). In operation, each of the helical inserts 540a-d directs air travelling through the respective adsorption bed 520a-d along a generally helical or spiral-shaped pathway (e.g., as shown by the air 12 travelling through the adsorption beds 520b and 520c in FIGS. 5B and 5C). As a result, the helical inserts 540a-d provide an air flow path through the respective adsorption bed that is longer than the length of the adsorption bed itself. Accordingly, utilizing the helical inserts 540a-d with the adsorption beds 520a-d effectively increases an aspect ratio (e.g., length to diameter) of the adsorption beds 520a-d without increasing the overall length of the adsorption bed. For example, using a helical insert can increase the aspect ratio of an adsorption bed by at least 10%, at least 20%, at least 30%, at least 40%, at least 50%, at least 100%, at least 200%, or greater. Without being bound by theory, this is expected to (1) increase, for a given adsorption bed size, the oxygen generated by the adsorption bed, and/or (2) enable use of a relatively more compact adsorption bed while maintaining oxygen generation capacity, which in turn can reduce the size of the oxygen generation assembly and thus the ventilator.

Referring first to FIG. 5A, the helical insert 540a can create one or more chambers, and thus, one or more flow pathways, within the adsorption bed 520a. In the illustrated embodiment, for example, the helical insert 540a creates a first chamber 544a₁ and a second chamber 544a₂ through the adsorption bed 520a. In other embodiments, the helical insert 540a can be configured to create at least three, four, five, or more chambers. Each of the chambers 544a can be fluidly isolated from each other, such that each of the chambers 544a can define a respective flow path through the interior 521a of the adsorption bed 520a. In the illustrated embodiment, for example, the first chamber 544a₁ defines a first flow path 542a₁ through the adsorption bed 520a and the second chamber 544a₂ defines a second flow path 542a₂ through the adsorption bed 520a. In embodiments in which the helical insert 540a creates more than two chambers, the helical insert 540a will also define more than two flow paths 542a (e.g., three, four, five, or more flow paths).

The helical insert 540a can be sized such that it has an outer circumference that fits within but is configured to abut or nearly abut an interior surface of the adsorption bed 520a. For example, the outer circumference of the helical insert 540a can be the same as or very slightly smaller than the inner circumference of the interior surface, such that the helical insert 540a has an outer dimension D1 (e.g., width, diameter, etc.) that is generally similar to or very slightly less than an inner dimension D2 (e.g., width, diameter, etc.) of the adsorption bed 520a. Because the helical insert 540a is dimensioned to fit snuggly within the adsorption bed 520a, the first chamber 544a₁ and the first flow path 542a₁ can be substantially or fully fluidly isolated from the second chamber 544a₂ and the second flow path 542a₂. In some embodiments, a portion of the helical insert 540a configured to abut the interior surface of the adsorption bed 520a can be sealed to (e.g., glued, taped, adhered to, etc.) the interior surface to further fluidly isolate the first flow path 542a₁ and the second flow path 542a₂.

In some embodiments, one or more of the chambers of the adsorption bed 520a can be connected in series, e.g., to form multiple passes within a single adsorption bed and/or to further increase the adsorption bed's aspect ratio. In at least some embodiments, for example, the first chamber 544a₁ can be fluidly coupled to the second chamber 544a₂ at an end (not shown) of the adsorption bed 520a, such that gases can enter the adsorption bed 520a flow through the first chamber 544a₁ via the first flow path 542a₁ and then flow through the second chamber 544a₂ via the second flow path 542a₂. The helical inserts 540b-d of FIGS. 5B-D can include chambers and flow paths generally similar to or the same as the chambers 544a and the flow paths 542a of the helical insert 540a.

The pitch of the helical inserts 540a-d can also be varied to achieve different aspect ratios and flow characteristics. For example, FIGS. 5B and 5C illustrate helical inserts 540b, 540c having different pitches (as used herein, the term “pitch” refers to a single complete rotation of a helical insert). In the illustrated embodiments, for example, the helical insert 540b has a first pitch P1 and the helical insert 540c has a second pitch P2 that is greater/tighter than the first pitch P1. The degree of pitch changes the effective length of the flow path extending through the adsorption bed. For example, for a helical insert and adsorption bed of a given length, a higher/tighter pitch corresponds to more rotations, a longer flow path, and thus a higher aspect ratio, whereas a smaller/looser pitch corresponds to fewer rotations, a shorter flow path, and thus a lower aspect ratio. Accordingly, in the illustrated embodiment, the adsorption bed 520b has a shorter flow path and thus a lower aspect ratio compared to the adsorption bed 520c (for a given length of the adsorption beds). The pitch of the helical inserts 540a-d can be selected based at least partially on a desired aspect ratio, design criteria of an associated ventilator or oxygen concentrator, manufacturing constraints of the associated adsorption bed, an amount of back pressure generated by the nitrogen-adsorbent material carried by the adsorption beds, etc. For a bed having a given length and diameter, a tighter pitch (e.g., FIG. 5C) will increase the length of the flow path and thus increase the backpressure generated by the nitrogen-adsorbent material, whereas a looser pitch (e.g., FIG. 5B) will decrease the length of the flow path and thus decrease the backpressure generated by the nitrogen-adsorbent material. Accordingly, the pitch can be selected to “fine tune” the backpressure generated by the nitrogen-adsorbent material to optimize the function of the adsorption bed. In some embodiments, the pitch can also be selected based on a compressor (not shown), e.g., to adjust the effective flow generated by a compressor without having to change a setting (e.g., speed) of the compressor itself.

In some embodiments, adsorption beds configured in accordance with embodiments of the present technology include helical inserts having an overall pitch VP that is varied or non-uniform, e.g., so as to include two or more differently-sized sub-pitches SP. Referring to FIG. 5D, the overall pitch VP of the helical insert 540d varies along a longitudinal axis of the adsorption bed 520d, e.g., from a first (e.g., air inflow) end 520d₁ of the adsorption bed 520d to a second (e.g., oxygen outflow) end 520d₂ of the adsorption bed 520d. In the illustrated embodiment, the overall pitch VP includes four sub-pitches SP1-SP4 (e.g., a first sub-pitch SP1, a second sub-pitch SP2, a third sub-pitch SP3, and a fourth sub-pitch SP4). The first sub-pitch SP1 is less than the second sub-pitch SP2, the second sub-pitch SP2 is less than the third sub-pitch SP3, and the third sub-pitch SP3 is less than the fourth sub-pitch SP4, such that the overall pitch VP increases along the longitudinal axis of the adsorption bed 520d from the first end 520d₁ toward the second end 520d₂. In other embodiments, the overall pitch VP can include more or fewer sub-pitches, such as at least two, three, five, six, or any other suitable number of number pitches. In these and other embodiments, individual ones of the sub-pitches can have any other suitable relationship to one or more of the other sub-pitches.

In operation, air (e.g., air 12 of FIG. 1) can enter the adsorption bed 520d via the first end 520d₁ and flow through the interior 521d toward the second end 520d₂. As the air flow toward the second end 520d₂, nitrogen-adsorbent material (e.g., the nitrogen-adsorbent material 429 of FIG. 4) within the interior 521d can preferentially adsorb nitrogen from the air which, in turn, reduces the amount of nitrogen in the air (as well as the volume of the air) as the air flows toward the second end 520d₂. Accordingly, the air at or near the second end 520d₂ is expected to have less nitrogen content than air at or near the first end 520d₁ and, in turn, a reduced quantity of nitrogen-adsorbent material can be positioned at or near the second end 520d₂ compared to the first end 520d₁. Moreover, the volume of air decreases as it flows from the first end 520d₁ toward the second end 520d₂. The overall pitch VP of the helical insert 540d can therefore be increased at or near the second end 520d₂, e.g., based at least partially on accommodating the reduced quantity of nitrogen-adsorbent material needed at or near the second end 520d₂ and/or the reduced volume of air that travels to the second end 520d₂.

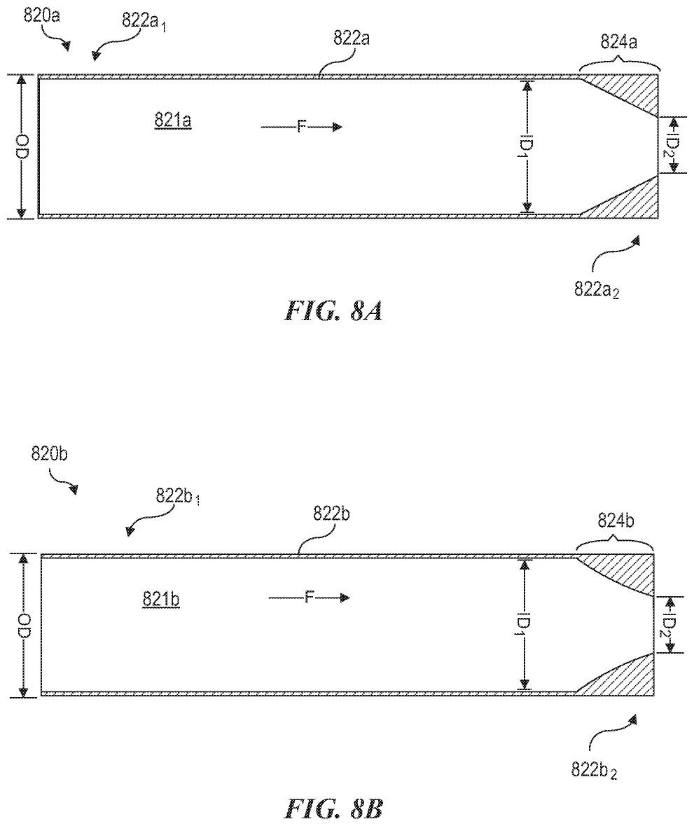

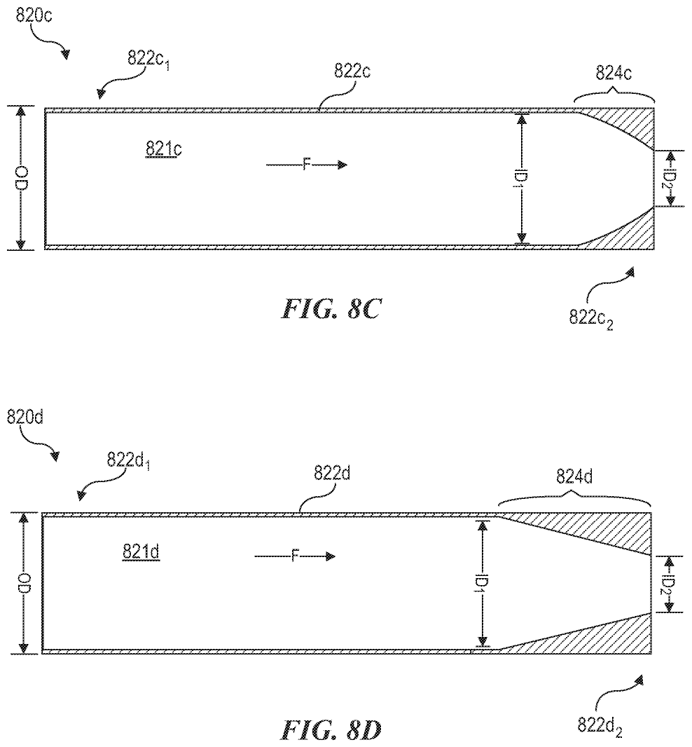

Varying the overall pitch VP of the helical insert 540d can result in the adsorption bed 520d operating generally similar to or the same as an adsorption bed that has a tapered or conical geometry (e.g., having one or more dimensions (width, diameter, cross-sectional area, etc.) that decreases along a length of the tapered/conical adsorption bed). Examples of adsorption beds with a tapered/conical geometry are described in greater detail with reference to FIGS. 8A-8C. In at least some embodiments, for example, the variation in the sub-pitches SP1-SP4 along the longitudinal axis of the helical insert 540d are selected such that the interior 521d of the adsorption bed 520d has a volume generally similar to or the same as an interior volume of a tapered/conical adsorption bed. Additionally, or alternatively, the increased relative pitch at or near the second end 520d₂ of the adsorption bed 520d can reduce the volume of nitrogen-adsorbent material carried by the adsorption bed 520d at or near the second end 520d₂; this reduction in volume of nitrogen-adsorbed material can be generally similar to or the same as the reduction in volume of nitrogen-adsorbent material in adsorption beds that are longitudinally tapered or conical. In some embodiments, the variable tapering can be designed such that the flow of gas moving through the adsorption bed 520d remains substantially constant. In such embodiments, the pitch is generally looser proximate the first end 520d₁ configured to receive the air 12 and tighter toward the second end 520d₂ configured to output oxygen because the volume of the air decreases as the air 12 moves from the first end 520d₁ toward the second end 520d₂ due to adsorption of nitrogen, as previously described.

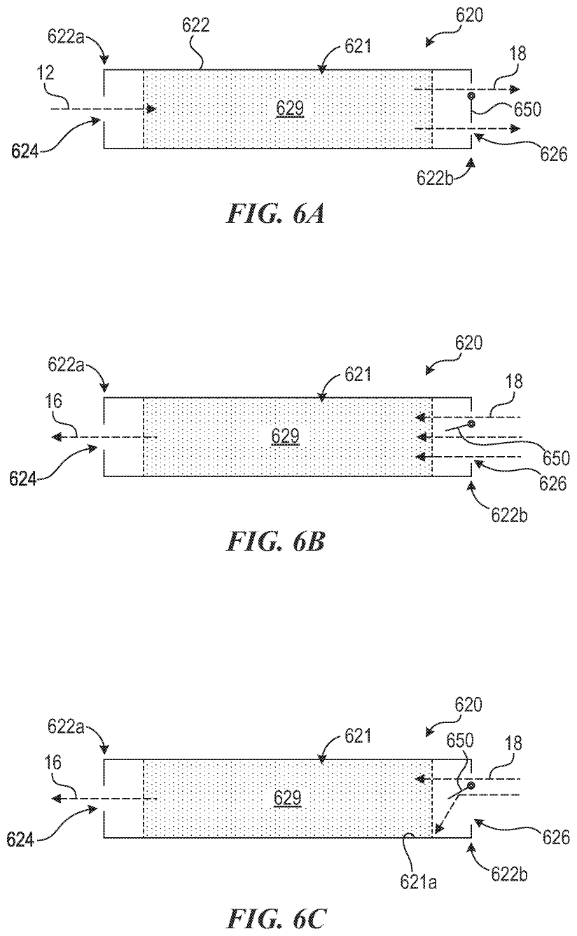

The present technology further includes adsorption beds with various features for improving performance of the adsorption bed during the adsorption phase and/or the purge phase of the oxygen generation process. FIGS. 6A-6C are a series of schematic views illustrating operation of an adsorption bed 620 during an oxygen generation process and configured in accordance with embodiments of the present technology. Specifically, FIG. 6A shows the adsorption bed 620 during an adsorption phase of the oxygen generation process, and FIGS. 6B and 6C illustrate the adsorption bed 620 during a desorption or purge phase of the oxygen generation process. The adsorption bed 620 can be generally similar to or the same as any of the adsorption beds described herein, or another suitable adsorption bed.

Referring to FIGS. 6A-6C together, the adsorption bed 620 can include a housing 622 having a first end 622a and a second end 622b opposite and spaced apart from the first end 622a. The housing 622 can include a first aperture or port 624 at the first end 622a and one or more second apertures or ports 626 at the second end 622b. The housing 622 can at least partially define an interior 621 of the adsorption bed 620. The interior 621 can be at least partially filled with nitrogen-adsorbent material 629. The second end 622b can include a valve 650 transitionable between a first (e.g., adsorption phase, closed, etc.) configuration (FIG. 6A) and a second (e.g., desorption phase, purge phase, open, etc.) configuration (FIGS. 6B and 6C). In the first (e.g., closed) configuration, gas can enter or exit the second end 622b of the adsorption bed solely by flowing through the second ports 626. In the second (e.g., open) configuration, the surface area of one or more of the second ports 626 available for gas ingress/egress is increased. The valve 650 can be a one-way valve, a flapper-type valve, or any other suitable valve. Although shown as described as a single valve 650 in FIGS. 6A-6C, in other embodiments adsorption beds of the present technology can include more valves 650, such as at least two, three, four, or any other suitable number of valves.

Referring to FIG. 6A, during the adsorption phase, the adsorption bed 620 can receive air 12 via the first port 624 at the first end 622a. The air 12 can flow through the interior 621 of the adsorption bed 620, and the nitrogen-adsorbent material 629 contained therein can preferentially adsorb nitrogen from the air 12, e.g., to generate concentrated oxygen 18. In the adsorption phase, the valve 650 can be in the first (e.g., closed) configuration and can at least partially restrict the flow of oxygen 32 through the second end 622b of the adsorption bed 620 (e.g., only permitting the flow of oxygen 18 to exit via the second ports 626) which, in turn, can maintain a relatively high pressure throughout all or part of the interior 621 of the adsorption bed 620.

Referring to FIG. 6B, during the desorption phase, the adsorption bed 620 can receive gas (e.g., oxygen 18 or another gas) via the second end 622b. The oxygen 18 can flow through the interior 621 of the adsorption bed 620, at least partially desorb nitrogen captured by the nitrogen-adsorbent material 629 during the adsorption phase, and flow out of the adsorption bed 620 as nitrogen-rich gas 16, e.g., to regenerate the nitrogen-adsorbent material 629 for at least one subsequent adsorption phase. In the desorption phase, the valve 650 can be in the second (e.g., open) configuration, providing a relatively larger area for fluid to flow through relative to when the valve 650 is in the first (e.g., closed) position. This is expected to provide an increased flow rate and/or an increased volume of gas flow through the second end 622b compared to the flow rate and/or volume of flow through the second end 622b when the valve 650 is in the first configuration (FIG. 6A). The increased flow rate and/or volume of oxygen 18 flow through the second end 622b can provide an increased surface area of flow during the desorption phase which, in turn, can increase the rate at which nitrogen is desorbed and the nitrogen-adsorbent material 629 is regenerated. Additionally, or alternatively, having the valve 650 in the second position during the desorption phase can decrease the pressure of the interior 621 of the adsorption bed 620, which can also increase the rate at which nitrogen is desorbed and the nitrogen-adsorbent material 629 is regenerated.

Referring to FIG. 6C, in some embodiments, the valve 650 can be configured to redirect or otherwise alter the flow of fluid through the second end 622b during the desorption phase. In the illustrated embodiment, for example, when the valve 650 is in the second configuration, the valve 650 can be configured to redirect a portion of the oxygen 18 toward at least one side wall 621a of the adsorption bed's interior 621. In other embodiments, the valve 650 can be configured to redirect the flow of oxygen 18 toward any other suitable portion of the adsorption bed's interior 621. Without being bound by theory, it is believed that adsorption beds including valves configured to redirect flow during the desorption phase can advantageously increase flow diffusion throughout all or part of the adsorption bed's interior and/or create a more uniform distribution of flow throughout all or part of the adsorption bed's interior.

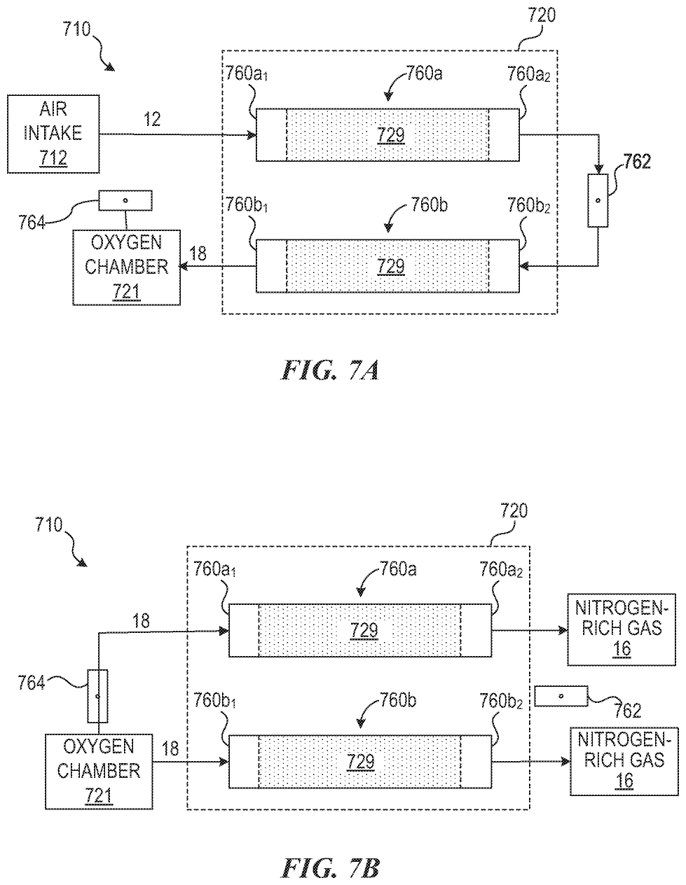

In some embodiments, adsorption beds of the present technology are configured to provide different flow paths during the adsorption phase and the purge phase. For example, FIGS. 7A and 7B are schematic illustrations of an oxygen assembly 710 including an adsorption bed 720 having variable flow paths and configured in accordance with embodiments of the present technology. Referring to FIGS. 7A and 7B collectively, the adsorption bed 720 includes a first section 760a containing nitrogen-adsorbent material 729 and a second section 760b containing nitrogen-adsorbent material 729. The first section 760a can have a first end 760a₁ and a second end 760a₂ opposite the first end 760a₁, and the second section 760b can likewise have a first end 760b₁ and a second end 760b₂ opposite the first end 760b₁. Each of the sections 760a,b can have a length less than, equal to, or greater than the length of the adsorption bed 720.

The first section 760a and the second section 760b can be separated by a flow control element 762, which can transition between an open position in which the flow control element 762 permits gas to flow between the first section 760a and the second section 760b (as shown in FIG. 7A) and a closed position in which the flow control element 762 prevents gas flow between the first section 760a and the second section 760b (as shown in FIG. 7B). Depending on the configuration of the flow control element 762, the sections 760a,b can either be coupled in series (e.g., as shown in FIG. 7A) or in parallel (e.g., as shown in FIG. 7B). The flow control element 762 can be a valve (e.g., a solenoid valve, a rotary valve, etc.) or other suitable feature that can selectively move to fluidly connect and fluidly isolate the first and second sections 760a,b.

The flow control element 762 is configured to cycle between the open position and the closed position during the oxygen generation cycle. For example, the flow control element 762 is generally in the open position during the adsorption phase of the oxygen generation cycle and in the closed position during the purge phase of the oxygen generation cycle. Referring to FIG. 7A, which shows the flow control element 762 in the open position during the adsorption phase, the adsorption bed 720 can receive air 12 via a patient air intake 712 or other suitable inlet at the first end 760a₁ of the first section 760a. The air 12 can flow through the first section 760a toward the second end 760a₂ of the first section 760a; as the air 12 flows through the first section 760a, the nitrogen-adsorbent material 729 contained therein can preferentially capture and retain (e.g., adsorb) nitrogen from the air 12. When the flow control element 762 is in the first configuration, the flow control element 762 can fluidly couple the second end 760a₂ of the first section 760a to the second end 760b₂ of the second section 760b, such that the air 12 flowing through the first section 760a can be directed by the flow control element 762 to and/or toward the second section 760b. The air 12 can continue to flow through the second section 760b and toward the first end 760b₁ of the second section 760b; as the air 12 flows through the second section 760b, the nitrogen-adsorbent material 729 contained therein can preferentially adsorb at least a portion of any nitrogen remaining in the air 12, e.g., to generate concentrated oxygen 18 for delivery to a patient. Because the first and second sections 760a, 760b are fluidly coupled by the flow control element 762 during the adsorption phase, the adsorption bed 720 can have an increased aspect ratio (e.g. length vs. width/diameter) during the adsorption phase.

Before and or during the transition between the adsorption phase and the purge phase, the flow control element 762 can be transitioned (e.g., rotated) from the first position in which the flow control element 762 fluidly couples the first and second sections 760a-b to the second configuration in which the flow control element 762 fluidly isolates (e.g., does not fluidly couple) the first and second sections 762a,b. Referring to FIG. 7B, which shows the flow control element 762 in the closed position during the desorption or purge phase, oxygen 18 (or another suitable fluid) can be supplied to the respective first ends 760a₁, 760b₁ of the first and second sections 760a, 760b to at least partially desorb or purge nitrogen captured by the nitrogen-adsorbent material 729 in each of the first and second sections 760a, 760b during the adsorption phase, e.g., to at least partially regenerate the nitrogen-adsorbent material 729. Because the flow control element 762 fluidly isolates the first and second sections 760a, 760b, the respective second ends 760a2, 760b₂ of the first and second sections 760a, 760b can allow nitrogen-rich gas 16 to flow out of the adsorption bed 720 during the purge phase (e.g., instead of directing the nitrogen-rich gas 16 to flow between the first section 760a and the second section 760b). Because the first and second sections 760a, 760b are fluidly isolated by the flow control element 762 during the purge phase, the adsorption bed 720 can have a decreased aspect ratio during the desorption phase, e.g., relative to the aspect ratio in the adsorption phase (FIG. 7A).

To facilitate the flow of oxygen 18 through both the first section 760a and the second section 760b during the desorption phase, the oxygen assembly 710 can further include an oxygen chamber valve 764 (also referred to simply as a “valve 764”). The oxygen chamber valve 764 can control flow through a pathway connecting the oxygen chamber 721 to the first section 760a. Accordingly, during the adsorption phase and as shown in FIG. 7A, the oxygen chamber valve 764 is in a closed position or otherwise blocks oxygen 18 from flowing into the first end 760a₁ of the first section 760a. However, during the desorption phase and as shown in FIG. 7B, the oxygen chamber valve 764 is in an open position or otherwise permits oxygen 18 to flow from the oxygen chamber 721 into the first end 760a₁ of the first section 760a. In some embodiments, the oxygen chamber valve 764 is a three-way valve that during the adsorption phase (1) enables air 12 to flow from the air intake 712 to the first section 760, and (2) blocks oxygen 18 from flowing from the oxygen chamber 721 to the first section, and during the desorption phase (3) blocks air 12 from flowing from the air intake 712 to the first section 760, and (4) enables oxygen 18 to flow from the oxygen chamber 721 to the first section 760.

Adsorption beds having variable flow paths for the adsorption phase and the purge phase enable the flow path to be optimized to the particular phase of the oxygen generation cycle. For example, the adsorption bed 720 is configured to have a relatively higher aspect ratio during the adsorption phase and a relatively lower aspect ratio during the purge phase. This is expected to provide several advantages. For example, it is beneficial for adsorption beds to have an increased aspect ratio during the adsorption phase to allow for increased nitrogen adsorption. It is also beneficial to have a reduced aspect ratio during the desorption or purge phase to allow for an increased regeneration rate for the nitrogen-adsorbent material. For example, a decreased aspect ratio is generally correlated with a decreased backpressure, decreased power drive requirements to flow gas through the adsorption bed, and an increased cycle time, all of which are advantageous during the desorption phase. Accordingly, adsorption beds configured in accordance with embodiments of the present technology can provide increased nitrogen adsorption during the adsorption phase and an increased nitrogen-adsorbent material regeneration rate during the purge phase.

In some embodiments, the present technology includes adsorption beds having a tapered shape such that an inner cross-sectional dimension of the adsorption bed is smaller at the product end than at the feed end of the bed. For example, FIGS. 8A-8D illustrate examples of adsorption beds having a tapered shape at the product end of the bed and configured in accordance with select embodiments of the present technology. More specifically, FIG. 8A is a longitudinal cross-sectional view of a first adsorption bed 820a having a first tapered shape and configured in accordance with select embodiments of the present technology, FIG. 8B is a longitudinal cross-sectional view of a second adsorption bed 820b having a second tapered shape and configured in accordance with select embodiments of the present technology, FIG. 8C is a longitudinal cross-sectional view of a third adsorption bed 820c having a third tapered shape and configured in accordance with select embodiments of the present technology, and FIG. 8D is a longitudinal cross-sectional view of a fourth adsorption bed 820d having a fourth tapered shape and configured in accordance with select embodiments of the present technology.

Referring to FIGS. 8A-8D collectively, each of the first adsorption bed 820a, the second adsorption bed 820b, the third adsorption bed 820c, and the fourth adsorption bed 820d (collectively referred to as the adsorption beds 820) includes a corresponding housing 822a-d defining an interior 821a-d for housing nitrogen-adsorbent material. Each housing 822a-d extends between a first end portion 822a₁-822d₁ and a second end portion 822a₂-822d₂. During the adsorption phase, air flows through the interior 821a-d between the first end portion 822a₁-822d₁ and the second end portion 822a₂-822d₂, in the direction indicated by the arrow F. During the desorption/purge phase, oxygen flows through the interior 821a-d in the opposite direction.

Each of the adsorption beds 820 includes a tapered portion 824a-d proximate the second end portion 822a₂-822d₂. Each of the tapered portions 824a-d tapers from a first inner diameter ID₁ to a second inner diameter ID₂. The first inner diameter ID₁ is greater than the second inner diameter ID₂, such that an inner diameter of the interior 821a-d decreases along the length of the tapered portion 824a-d. In some embodiments, the first inner diameter ID₁ can be between about 1.25 inches and about 2.25 inches, or between about 1.5 inches and about 2 inches, or about 1.75 inches, and the second inner diameter ID₂ can be between about 0.25 inch and 1.25 inches, or between about 0.5 inch and 1 inch, or about 0.75 inch. In some embodiments, the ratio between the first inner diameter ID₁ and the second inner diameter ID₂ can be between about 5:1 and 1.5:1, or between about 4:1 and 1.5:1, or between about 3:1 and about 2:1, or about 2.5:1. The foregoing ranges, dimensions, and ratios are provided by way of example only—in some embodiments, the first inner diameter ID₁ and/or the second inner diameter ID₂ can have dimensions and ratios outside the foregoing ranges. Of note, the outer diameter OD of the housing 822a-d remains substantially the same, such that the adsorption beds 820 retain their generally cylindrical outer profile.

The shape of the tapered portions 824a-d differ between the adsorption beds 820. For example, as shown in FIG. 8A, the tapered portion 824a of the first adsorption bed 820a is linearly tapered, such that the decrease in diameter between the first inner diameter ID₁ and the second inner diameter ID₂ is generally constant along the length of the tapered portion 824a. As shown in FIG. 8B, the tapered portion 824b of the second adsorption bed 820b has a convex tapering, such that the change in diameter along the length of the tapered portion 824b is not constant. Similarly, as shown in FIG. 8C, the tapered portion 824c of the third adsorption bed 820c has a concave tapering, such that the change in diameter along the length of the tapered portion 824c is also not constant. As shown in FIG. 8D, the tapered portion 824d of the fourth adsorption bed 820d is linearly tapered like the first adsorption bed 820a of FIG. 8A, but has a longer length than the tapered portion 824a of the first adsorption bed 820a such that the rate of change in the inner diameter of the tapered portion 824d is less.

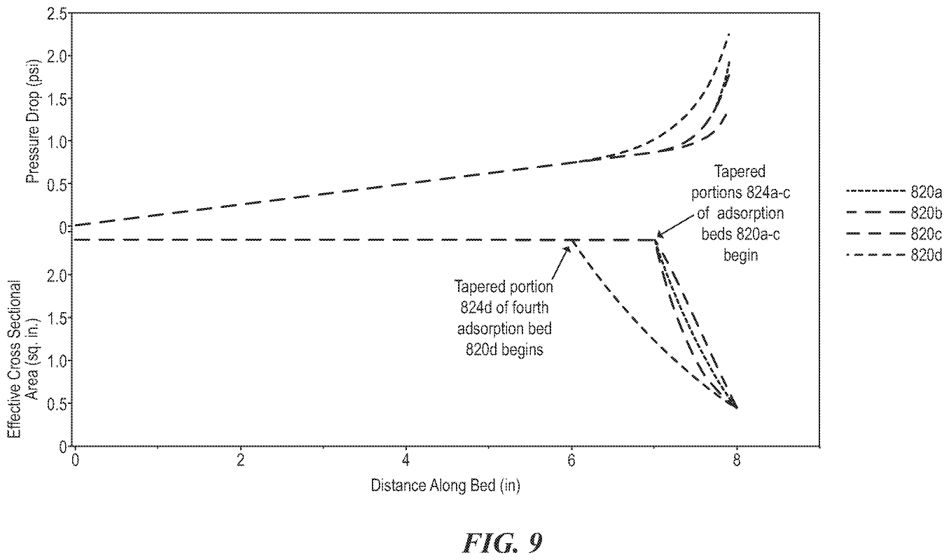

The length and the shape of the tapered portion 824a-d can impact the pressure and flow characteristics, and thus the adsorbent characteristics, of the adsorption beds 820. For example, FIG. 9 is a graph showing (1) a pressure drop along a length of each adsorption bed 820 for a typical flow during an adsorption phase, and (2) the effective cross-sectional area of each adsorption bed 820 along its length. As shown, each of the tapered portions 824a-d of each adsorption bed 820 has a slightly different effective cross-sectional area, and thus has a slightly different pressure drop during operation. Thus, the length and/or shape of the tapered portions 824a-d can be selected based on desired pressure and/or flow characteristics of the adsorbent bed. As one skilled in the art will appreciate, the adsorbent beds 820 in FIGS. 8A-8D provide four representative examples of different shapes and sizes for tapered portions. However, the present technology is not limited to the shapes and sizes shown in FIGS. 8A-8D. Indeed, as one skilled in the art will appreciate from the disclosure herein, the tapered portions can have other suitable shapes and sizes beyond those expressly shown herein without deviating from the scope of the present technology. For example, the tapered portions can have different geometries (e.g., step geometries, irregular geometries, etc.), different lengths, different degrees of tapering, and/or different inner dimensions.

Without intending to be bound by theory, and as shown in FIG. 9, the tapered portion 824a-d causes the average velocity within the adsorbent bed 820a-d to increase at an increasing rate at the second end portion 822a₂-822d₂ (e.g., the product end). Without intending to be bound by theory, this is expected to reduce the rate of change of oxygen concentration produced by the adsorption bed 820. That is, incorporating the tapered portion 824a-d into an adsorption bed is expected to increase an active length of the mass transfer zone and thus stabilize the concentration of oxygen flowing out of the second end portion 822a₂-822d₂ of the adsorption bed. Accordingly, in some embodiments adsorption beds having a tapered portion at the product end of the bed are expected to provide a more consistent output of oxygen.

The tapered portions 824a-d can either be built into the adsorbent bed itself (e.g., as part of the housing 822a-d), or can be a separate component (e.g., an insert) that a user can install in the adsorbent bed. In embodiments in which the tapered portion 824a-d comprises an insert, a user can repeatedly change which insert is being used to adjust the dimensions of the flow path, e.g., to achieve a desired oxygen output.

### EXAMPLES

Several aspects of the present technology are set forth in the following examples:

1. A ventilator system with integrated oxygen production, the ventilator system comprising:
a housing;
one or more air intakes in the housing configured to receive air from external to the housing;
a ventilation assembly positioned within the housing and configured to provide the received air to a ventilation delivery circuit during an inspiratory phase of a breath;
an oxygen assembly positioned within the housing and configured to provide concentrated oxygen to an oxygen delivery circuit during the inspiratory phase of the breath, wherein the oxygen assembly includes:
   an adsorption bed assembly having—
      an adsorption bed configured to generate the concentrated oxygen from the received air, and
      an integrated panel coupled to an end portion of the adsorption bed,
      wherein the adsorption bed assembly is removable from the housing by a user, and wherein, when the adsorption bed assembly is positioned within the housing, the integrated panel forms a portion of an exterior surface of the housing.

2. The ventilator system of example 1 wherein the adsorption bed assembly further comprises an attachment mechanism configured to releasably couple the integrated panel to the housing.

3. The ventilator system of example 2 wherein the attachment mechanism includes a latching member pivotally coupled to the integrated panel, and wherein the latching member is pivotably moveable between a first position in which the integrated panel is locked to the housing and a second position in which the integrated panel is unlocked from the housing.

4. The ventilator system of example 2 wherein the attachment mechanism includes (i) a keyhole rotatable between a locked position and an unlocked position, and (ii) a wire bail moveable between a locked position and an unlocked position, and wherein the attachment mechanism is configured such that, to disengage the adsorption bed assembly from the housing, both the keyhole and the wire bail must be in the unlocked position.

5. The ventilator system of any of examples 1-4 wherein the adsorption bed assembly includes a first end portion having one or more first airflow ports configured to receive the received air and a second end portion having one or more second airflow ports configured to output the concentrated oxygen.

6. The ventilator system of example 5 wherein the integrated panel includes the one or more second airflow ports.

7. The ventilator system of example 6 wherein the integrated panel further includes a fluid transfer area fluidically coupling the adsorption bed and the one or more second airflow ports.

8. The ventilator system of any of examples 5-7 wherein the one or more first airflow ports have corresponding one or more first airflow axes and the one or more second airflow ports have one or more corresponding second airflow axes, and wherein the one or more first airflow axes are parallel to the one or more second airflow axes.

9. The ventilator system of any of examples 5-7 wherein the one or more first airflow ports have corresponding one or more first airflow axes and the one or more second airflow ports have one or more corresponding second airflow axes, and wherein the one or more first airflow axes are substantially perpendicular to the one or more second airflow axes.

10. An adsorption bed, comprising:
a housing defining an interior of the adsorption bed, the interior of the adsorption bed defining an inner dimension of the adsorption bed; and
an elongated helical insert positioned within the housing, wherein the elongated helical insert has an outer dimension that is about the same as the inner dimension of the adsorption bed,
wherein the helical insert defines a first flow path through the interior and a second flow path different than the first flow path through the interior, wherein the first flow path and the second flow path are fluidically isolated along a substantial portion of their respective lengths.

11. The adsorption bed of example 10 wherein the first flow path has a first length, the second flow path has a second length, and the adsorption bed has a third length, and wherein the third length is less than the first length and the second length.

12. The adsorption bed of example 11 wherein the first length is approximately the same as the second length.

13. The adsorption bed of any of examples 10-12 wherein inner dimension is an inner diameter of the adsorption bed, and wherein the outer dimension is an outer diameter of the helical insert.

14. The adsorption bed of any of examples 10-13 wherein the helical insert has a constant pitch.

15. The adsorption bed of any of examples 10-14 wherein the helical insert has a variable pitch that changes along a longitudinal axis of the helical insert.

16. The adsorption bed of example 15 wherein the variable pitch includes a first pitch proximate a first end of the adsorption bed that is configured to receive air, and a second pitch proximate a second end of the adsorption bed opposite the first end and that is configured to output concentrated oxygen, and wherein the second pitch is greater than the first pitch.

17. The adsorption bed of any of examples 10-16 wherein an end region of the first flow path is fluidly coupled to an end region of the second flow path such that the first flow path and the second flow path are fluidly coupled in series.

18. The adsorption bed of example 17, further comprising a valve positioned between the first flow path and the second flow path, wherein the valve is transitionable between (i) a first configuration in which the valve does not prevent gas from flowing between the first flow path and the second flow path, and (ii) a second configuration in which the valve substantially prevents gas from flowing between the first flow path and the second flow path.

19. An adsorption bed, comprising:
a housing defining an interior of the adsorption bed, wherein the interior includes a first flow path and a second flow path; and
a valve positioned between the first flow path and the second flow path, wherein the valve is transitionable between (i) a first configuration in which the first flow path and the second flow path are fluidly connected, and (ii) a second configuration in which the first flow path and the second flow path and fluidly isolated,
wherein the valve is configured to be in the first configuration during an adsorption phase of an oxygen generation cycle and the second configuration during a desorption phase of the oxygen generation cycle.

20. The adsorption bed of example 19 wherein the first flow path has a first length, the second flow path has a second length, and the adsorption bed has a third length, and wherein the sum of the first length and the second length is greater than the third length.

21. The adsorption bed of example 19 or example 20 wherein the adsorption bed has a first aspect ratio when the valve is in the first configuration and a second aspect ratio less than the first aspect ratio when the valve is in the second configuration.

22. An adsorption bed, comprising:
a housing;
a desiccant material portion within the housing and configured to contain a desiccant material;
a nitrogen-adsorbent material portion within the housing and configured to contain a nitrogen-adsorbent material; and
a valve positioned within the housing between the desiccant material portion and the nitrogen-adsorbent material portion, wherein the valve is transitionable between a first configuration in which the desiccant material portion is fluidly coupled to the nitrogen-adsorbent material portion and a second configuration in which the desiccant material portion is fluidly isolated from the nitrogen-adsorbent material portion.

23. The adsorption bed of example 22 wherein valve is configured to be in the first configuration when the adsorption bed is in use and in the second configuration when the adsorption bed is not in use.

24. The adsorption bed of example 22 or example 23 wherein, when the valve is in the second configuration, the valve is configured to prevent moisture captured within the desiccant material portion from migrating into the nitrogen-adsorbent material portion.

25. An adsorption bed, comprising:
a housing having a first end, a second end opposite the first end, and an interior extending between the first end and the second end;
one or more first apertures at the first end;
one or more second apertures at the second end; and
a valve positioned at the second end, wherein the valve is transitionable between (i) a first configuration in which the valve provides a first fluid resistance through the one or more second apertures, and (ii) a second configuration in which the valve provides a second fluid resistance through the one or more second apertures, the second fluid resistance being less than the first fluid resistance,
wherein the valve is configured to be in the first configuration when gas flows from the first end toward the second end during an adsorption phase of an oxygen generation cycle, and in the second configuration when gas flows from the second end toward the first end during a desorption phase of the oxygen generation cycle.

26. The adsorption bed of example 25 wherein, in the second configuration, the valve redirects at least a portion of the fluid flowing through the second end toward at least a sidewall of the interior.

27. The adsorption bed of example 25 or example 26 wherein the interior has a first pressure when the valve is in the first configuration and a second pressure less than the first pressure when the valve is in the second configuration.

28. An adsorption bed, comprising:
an interior configured to house a nitrogen-adsorbent material;
a seal assembly positioned at an end portion of the adsorption bed, the seal assembly including—
   a first sealing element extending at least partially around an outer perimeter of the seal assembly,
   a second sealing element extending at least partially around the outer perimeter of the seal assembly,
   a fluid channel defined between the first sealing element and the second sealing element and extending at least partially around the outer perimeter of the seal assembly, and
   one or more flow paths fluidly coupling the interior and the fluid channel,
wherein the adsorption bed is configured to be inserted into a corresponding housing within a system for generating concentrated oxygen, and wherein the adsorption bed is configured such that, when inserted into the corresponding housing of the system, the fluid channel aligns with one or more ports in the housing.

29. An adsorption bed, comprising:
a cylindrical housing extending between a first end portion and a second end portion and having an interior configured to house a nitrogen-adsorbent material,
wherein the adsorption bed includes a tapered portion proximate the second end portion, and
wherein an inner dimension of the interior decreases along the tapered portion and an outer dimension of the cylindrical housing remains substantially the same along the tapered portion.

30. The adsorption bed of example 29 wherein the cylindrical housing includes the tapered portion.

31. The adsorption bed of example 29 wherein the adsorption bed includes a tapered portion insert, and wherein the tapered portion insert is positioned within the interior of the cylindrical housing to define the tapered portion.

32. The adsorption bed of any of examples 29-31 wherein the tapered portion has a linear tapering.

33. The adsorption bed of any of examples 29-32 wherein the tapered portion has a convex tapering.

34. The adsorption bed of any of examples 29-33 wherein the tapered portion has a concave tapering.

35. The adsorption bed of any of examples 29-34 wherein the inner dimension is an inner diameter.

36. The adsorption bed of example 35 wherein the inner diameter decreases from a first inner diameter at a first end of the tapered portion to a second inner diameter at a second end of the tapered portion.

37. The adsorption bed of example 36 wherein the first inner diameter is between about 1.25 inches and 2.25 inches, and wherein the second inner diameter is between about 0.25 inch and about 1.25 inches.

38. The adsorption bed of example 36 or example 37 wherein a ratio between the first inner diameter and the second inner diameter is between about 5:1 and 1.5:1.

### IV. Conclusion

As one of skill in the art will appreciate from the disclosure herein, various components of the systems described above can be combined into a single system, device, or component. For example, an adsorption bed and/or oxygen assembly configured in accordance with the present technology may include any combination of the features described herein, including valves that isolate desiccant material from nitrogen-adsorbent material (e.g., as described with respect to FIG. 4), helical inserts to optimize aspect ratio (e.g., as described with respect to FIGS. 5A-5D), valving that changes the size of inflow/outflow ports (e.g., as described with respect to FIGS. 6A-6C), flow control elements that change the flow path during various phases of the oxygen generation cycle (e.g., as described with respect to FIGS. 7A and 7B), and/or tapered portions that change an inner dimension of adsorbent beds (e.g., as described with respect to FIGS. 8A-9). Likewise, any of the foregoing features can be incorporated into the ventilators described herein with reference to FIGS. 1-3B, into other ventilators with integrated oxygen production, or into standalone oxygen concentrators. As one skilled in the art will further appreciate, various components of the systems described above can be omitted, and/or additional components not explicitly described above may be added to the systems without deviating from the scope of the present technology. For example, it will be appreciated that specific embodiments of the technology have been described herein for purposes of illustration, but well-known structures and functions have not been shown or described in detail to avoid unnecessarily obscuring the description of the embodiments of the technology. Moreover, although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments. Accordingly, the present technology is not limited to the configurations expressly identified herein, but rather encompasses variations and alterations of the described systems and methods.

Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Further, where specific integers are mentioned herein which have known equivalents in the art to which the embodiments relate, such known equivalents are deemed to be incorporated herein as if individually set forth.

I/we claim:

1. A ventilator system with integrated oxygen production, the ventilator system comprising:
a housing;
one or more air intakes in the housing configured to receive air from external to the housing;
a ventilation assembly positioned within the housing and configured to provide the received air to a ventilation delivery circuit during an inspiratory phase of a breath;
an oxygen assembly positioned within the housing and configured to provide concentrated oxygen to an oxygen delivery circuit during the inspiratory phase of the breath, wherein the oxygen assembly includes:
an adsorption bed assembly having—
an adsorption bed configured to generate the concentrated oxygen from the received air, and
an integrated panel coupled to an end portion of the adsorption bed,
wherein the adsorption bed assembly is removable from the housing by a user, and wherein, when the adsorption bed assembly is positioned within the housing, the integrated panel forms a portion of an exterior surface of the housing.

2. The ventilator system of claim 1 wherein the adsorption bed assembly further comprises an attachment mechanism configured to releasably couple the integrated panel to the housing.

3. The ventilator system of claim 2 wherein the attachment mechanism includes a latching member pivotally coupled to the integrated panel, and wherein the latching member is pivotably moveable between a first position in which the integrated panel is locked to the housing and a second position in which the integrated panel is unlocked from the housing.

4. The ventilator system of claim 2 wherein the attachment mechanism includes (i) a keyhole rotatable between a locked position and an unlocked position, and (ii) a wire bail moveable between a locked position and an unlocked position, and wherein the attachment mechanism is configured such that, to disengage the adsorption bed assembly from the housing, both the keyhole and the wire bail must be in the unlocked position.

5. The ventilator system of claim 1 wherein the adsorption bed assembly includes a first end portion having one or more first airflow ports configured to receive the received air and a second end portion having one or more second airflow ports configured to output the concentrated oxygen.

6. The ventilator system of claim 5 wherein the integrated panel includes the one or more second airflow ports.

7. The ventilator system of claim 6 wherein the integrated panel further includes a fluid transfer area fluidically coupling the adsorption bed and the one or more second airflow ports.

8. The ventilator system of claim 5 wherein the one or more first airflow ports have corresponding one or more first airflow axes and the one or more second airflow ports have one or more corresponding second airflow axes, and wherein the one or more first airflow axes are parallel to the one or more second airflow axes.

9. The ventilator system of claim 5 wherein the one or more first airflow ports have corresponding one or more first airflow axes and the one or more second airflow ports have one or more corresponding second airflow axes, and wherein the one or more first airflow axes are substantially perpendicular to the one or more second airflow axes.

10. An adsorption bed, comprising:
a housing defining an interior of the adsorption bed, the interior of the adsorption bed defining an inner dimension of the adsorption bed; and
an elongated helical insert positioned within the housing, wherein the elongated helical insert has an outer dimension that is about the same as the inner dimension of the adsorption bed,
wherein the helical insert defines a first flow path through the interior and a second flow path different than the first flow path through the interior, wherein the first flow path and the second flow path are fluidically isolated along a substantial portion of their respective lengths.

11. The adsorption bed of claim 10 wherein the first flow path has a first length, the second flow path has a second length, and the adsorption bed has a third length, and wherein the third length is less than the first length and the second length.

12. The adsorption bed of claim 11 wherein the first length is approximately the same as the second length.

13. The adsorption bed of claim 10 wherein inner dimension is an inner diameter of the adsorption bed, and wherein the outer dimension is an outer diameter of the helical insert.

14. The adsorption bed of claim 10 wherein the helical insert has a constant pitch.

15. The adsorption bed of claim 10 wherein the helical insert has a variable pitch that changes along a longitudinal axis of the helical insert.

16. The adsorption bed of claim 15 wherein the variable pitch includes a first pitch proximate a first end of the adsorption bed that is configured to receive air, and a second pitch proximate a second end of the adsorption bed opposite the first end and that is configured to output concentrated oxygen, and wherein the second pitch is greater than the first pitch.

17. The adsorption bed of claim 10 wherein an end region of the first flow path is fluidly coupled to an end region of the second flow path such that the first flow path and the second flow path are fluidly coupled in series.

18. The adsorption bed of claim 17, further comprising a valve positioned between the first flow path and the second flow path, wherein the valve is transitionable between (i) a first configuration in which the valve does not prevent gas from flowing between the first flow path and the second flow path, and (ii) a second configuration in which the valve substantially prevents gas from flowing between the first flow path and the second flow path.

19. An adsorption bed, comprising:
a housing defining an interior of the adsorption bed, wherein the interior includes a first flow path and a second flow path; and
a valve positioned between the first flow path and the second flow path, wherein the valve is transitionable between (i) a first configuration in which the first flow path and the second flow path are fluidly connected, and (ii) a second configuration in which the first flow path and the second flow path and fluidly isolated,
wherein the valve is configured to be in the first configuration during an adsorption phase of an oxygen generation cycle and the second configuration during a desorption phase of the oxygen generation cycle.

20. The adsorption bed of claim 19 wherein the first flow path has a first length, the second flow path has a second length, and the adsorption bed has a third length, and wherein the sum of the first length and the second length is greater than the third length.

21. The adsorption bed of claim 19 wherein the adsorption bed has a first aspect ratio when the valve is in the first configuration and a second aspect ratio less than the first aspect ratio when the valve is in the second configuration.

22. An adsorption bed, comprising:
a housing;
a desiccant material portion within the housing and configured to contain a desiccant material;
a nitrogen-adsorbent material portion within the housing and configured to contain a nitrogen-adsorbent material; and
a valve positioned within the housing between the desiccant material portion and the nitrogen-adsorbent material portion, wherein the valve is transitionable between a first configuration in which the desiccant material portion is fluidly coupled to the nitrogen-adsorbent material portion and a second configuration in which the desiccant material portion is fluidly isolated from the nitrogen-adsorbent material portion.

23. The adsorption bed of claim 22 wherein valve is configured to be in the first configuration when the adsorption bed is in use and in the second configuration when the adsorption bed is not in use.

24. The adsorption bed of claim 22 wherein, when the valve is in the second configuration, the valve is configured to prevent moisture captured within the desiccant material portion from migrating into the nitrogen-adsorbent material portion.

25. An adsorption bed, comprising:
a housing having a first end, a second end opposite the first end, and an interior extending between the first end and the second end;
one or more first apertures at the first end;
one or more second apertures at the second end; and
a valve positioned at the second end, wherein the valve is transitionable between (i) a first configuration in which the valve provides a first fluid resistance through the one or more second apertures, and (ii) a second configuration in which the valve provides a second fluid resistance through the one or more second apertures, the second fluid resistance being less than the first fluid resistance,
wherein the valve is configured to be in the first configuration when gas flows from the first end toward the second end during an adsorption phase of an oxygen generation cycle, and in the second configuration when gas flows from the second end toward the first end during a desorption phase of the oxygen generation cycle.

26. The adsorption bed of claim 25 wherein, in the second configuration, the valve redirects at least a portion of the fluid flowing through the second end toward at least a sidewall of the interior.

27. The adsorption bed of claim 25 wherein the interior has a first pressure when the valve is in the first configuration and a second pressure less than the first pressure when the valve is in the second configuration.

28. An adsorption bed, comprising:
an interior configured to house a nitrogen-adsorbent material;
a seal assembly positioned at an end portion of the adsorption bed, the seal assembly including—
a first sealing element extending at least partially around an outer perimeter of the seal assembly,
a second sealing element extending at least partially around the outer perimeter of the seal assembly,
a fluid channel defined between the first sealing element and the second sealing element and extending at least partially around the outer perimeter of the seal assembly, and
one or more flow paths fluidly coupling the interior and the fluid channel,
wherein the adsorption bed is configured to be inserted into a corresponding housing within a system for generating concentrated oxygen, and wherein the adsorption bed is configured such that, when inserted into the corresponding housing of the system, the fluid channel aligns with one or more ports in the housing.

29. An adsorption bed, comprising:
a cylindrical housing extending between a first end portion and a second end portion and having an interior configured to house a nitrogen-adsorbent material,
wherein the adsorption bed includes a tapered portion proximate the second end portion, and
wherein an inner dimension of the interior decreases along the tapered portion and an outer dimension of the cylindrical housing remains substantially the same along the tapered portion.

30. The adsorption bed of claim 29 wherein the cylindrical housing includes the tapered portion.

31. The adsorption bed of claim 29 wherein the adsorption bed includes a tapered portion insert, and wherein the tapered portion insert is positioned within the interior of the cylindrical housing to define the tapered portion.

32. The adsorption bed of claim 29 wherein the tapered portion has a linear tapering.

33. The adsorption bed of claim 29 wherein the tapered portion has a convex tapering.

34. The adsorption bed of claim 29 wherein the tapered portion has a concave tapering.

35. The adsorption bed of claim 29 wherein the inner dimension is an inner diameter.

36. The adsorption bed of claim 35 wherein the inner diameter decreases from a first inner diameter at a first end of the tapered portion to a second inner diameter at a second end of the tapered portion.

37. The adsorption bed of claim 36 wherein the first inner diameter is between about 1.25 inches and 2.25 inches, and wherein the second inner diameter is between about 0.25 inch and about 1.25 inches.

38. The adsorption bed of claim 36 wherein a ratio between the first inner diameter and the second inner diameter is between about 5:1 and 1.5:1.
